(19) United States
(12) Patent Application Publication
(10) Pub. No.: US 2026/0284018 A1
(43) Pub. Date: Sep. 24, 2026

(54) DUAL INHIBITORS OF DYRK1A AND 5-HT2 FOR TREATING A BRAIN DISORDER

(71) Applicant: THE REGENTS OF THE UNIVERSITY OF CALIFORNIA, Oakland, CA (US)

(72) Inventors: David E. OLSON, Oakland, CA (US); Hunter WARREN, Oakland, CA (US)

(21) Appl. No.: 19/473,734

(22) PCT Filed: Apr. 17, 2024
(86) PCT No.: PCT/US2024/024911

§ 371 Date: Oct. 8, 2025

Related U.S. Application Data
(60) Provisional application No. 63/496,757, filed on Apr. 18, 2023.

(51) Int. Cl. A61K 31/437; A61K 31/19; A61K 31/357; A61K 31/428; A61K 31/496; A61K 31/519; A61K 31/53; A61K 31/55; A61K 31/551; A61K 31/5513; A61K 31/554; A61K 33/00

(52) U.S. Cl. CPC A61K 31/437; A61K 31/19; A61K 31/357; A61K 31/428; A61K 31/496; A61K 31/519; A61K 31/53; A61K 31/55; A61K 31/551; A61K 31/5513; A61K 31/554; A61K 33/00

(57) ABSTRACT

Provided herein are compounds that function as modulators of the kinase DYRK1A and serotonin 2C receptor (5-HT2CR).

### CROSS-REFERENCES TO RELATED APPLICATIONS

This application claims priority to U.S. Provisional Application No. 63/496,757, filed Apr. 18, 2023, which is incorporated herein in its entirety for all purposes.

### BACKGROUND

Compounds have been synthesized that function as both inhibitors of the kinase DYRK1A and serotonin 2C receptor (5-HT2CR) antagonists. These compounds are believed to have potential application as therapeutics for treating neurodegenerative diseases such as Alzheimer's Disease (AD). DYRK1A is known to phosphorylate both amyloid precursor protein and tau protein, both of which are implicated in the pathophysiology of AD and related neurodegenerative diseases. Inhibition of DYRK1A activity has been shown to prevent the development of AD-like phenotypes in vivo. In addition, antagonism of 5-HT2CRs has been shown to block stress-induced tau hyperphosphorylation in the hippocampus of mice, lending to the idea that inhibition of DYRK1A and antagonism of 5-HT2CRs may act synergistically in preventing and/or treating AD and related dementias.

### BRIEF SUMMARY OF THE INVENTION

In one embodiment, the present invention provides a method of treating a brain disorder, comprising administering to a subject in need thereof a therapeutically effective amount of a compound of Formula I or Formula II:

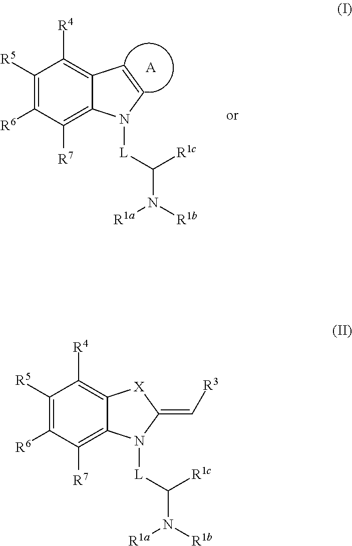

or a pharmaceutically acceptable salt thereof, in an amount sufficient to increase neuronal plasticity of the neuronal cell, wherein:
R^{1a}, R^{1b}, and R^{1c} are each independently hydrogen, C₁₋₆ alkyl, C₃₋₈ cycloalkyl, or C₄₋₁₄ alkyl-cycloalkyl;
alternatively, two of R^{1a}, R^{1b}, and R^{1c} are combined with the atoms to which they are attached to form a C₃₋₁₂ heterocycloalkyl;
Ring A is a 5 to 6 membered heteroaryl ring having 1 to 3 heteroatoms each independently N, O, or S, substituted with 0 to 3 R² groups;
R², R⁴, R⁵, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
alternatively, R⁴ and R⁵, R⁵ and R⁶, or R⁶ and R⁷ are combined with the atoms to which they are each attached to form a C₃₋₆ cycloalkyl, C₃₋₆ heterocycloalkyl, C₆₋₁₂ aryl, or C₅₋₁₀ heteroaryl; and
R³ is C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{8a} is C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{8b}, R^{8c} and R^{8d} are each independently H or C₁₋₆ alkyl;
X is O or S; and
L is C₁₋₆ alkylene.

In another embodiment, the present invention provides a method of treating a brain disorder, comprising administering to a subject in need thereof a therapeutically effective amount of a DYRK1A modulator and a 5-HT2 modulator, wherein each modulator is present in a sub-therapeutic amount.

In another embodiment, the present invention provides a method of treating a brain disorder, comprising administering to a subject in need thereof a therapeutically effective amount of a compound having a DYRK1A binding moiety and a 5-HT binding moiety.

### BRIEF DESCRIPTION OF THE DRAWINGS

FIG. 1 shows the synthetic route to harmine-based dual ligands HTW_A_94, HTW_A_200, and HTW_A_8.

FIG. 2 shows the synthetic route to TG003-based dual ligands HTW_B_14, HTW_B_26, and HTW_A_229.

FIG. 3 shows HTW_A_8 and HTW_B_14 are DYRK1A inhibitors. Both HTW_A_8 fumarate and HTW_B_14 fumarate are able to inhibit DYRK1A activity. All other compounds tested exhibited IC50 values of >10 μM.

FIG. 4 shows HTW_A_8 fumarate and HTW_B_14 fumarate are selective for 5-HT2C over 5-HT2A. Radioligand binding assays demonstrate that both HTW_A_8 fumarate and HTW_B_14 fumarate are higher affinity ligands for 5-HT2C than 5-HT2A.

FIG. 5 shows all dual-ligands are predicted to be non-hallucinogenic 5-HT2A ligands using psychLight. All compounds tested failed to activate psychLight in agonist mode but caused a significant decrease in sensor activation in antagonist mode. 5-HT=serotonin; KETSN=ketanserin.

FIG. 6 shows HTW_A_8 fumarate and HTW_B_14 fumarate are 5-HT2C antagonists in IP-One assays. Both HTW_A_8 fumarate and HTW_B_14 fumarate displayed no activation of 5-HT2A or 5-HT2C in the IP-One assay ran in agonist mode. Both compounds displayed antagonist activity in the assay, with moderate selectivity for antagonism of 5-HT2C.

FIG. 7 shows HTW_A_8 fumarate and HTW_B_14 fumarate do not produce a head-twitch response in mice. Both HTW_A_8 fumarate and HTW_B_14 fumarate failed to produce a head-twitch response in mice up to 30 mg/kg.

FIG. 8 shows HTW_A_8 fumarate can dampen the head-twitch response produced by 5-MeO-DMT. A 30 mg/kg pretreatment of HTW_A_8 fumarate was sufficient to dampen the head-twitch response produced by a 10 mg/kg treatment of 5-MeO-DMT.

FIG. 9 shows HTW_A_8 fumarate and HTW_B_14 fumarate do not significantly affect locomotion in mice. Both HTW_A_8 fumarate and HTW_B_14 fumarate do not significantly affect the total distance traveled by mice in an open field chamber at doses up to 30 mg/kg.

FIG. 10 shows HTW_A_8 fumarate can reduce levels of phospho-Tau in cortical neurons produced by an okadaic acid insult. A 50 μM pretreatment of HTW_A_8 fumarate can block an increase in Tau phosphorylated at Ser202/Thr205 induced by an okadaic acid insult in rat cortical neurons. This effect was not observed when pretreating with HTW_B_14 fumarate and there was no reduction in Tau phosphorylated at Ser396 with either compound. OKA=30 nM okadaic acid.

FIG. 11 shows neurite outgrowth assay conducted using primary rat cortical neurons. Neurons were treated with the MAO-A inhibitors toloxatone and harmaline, the DYRK1A inhibitors TG003 and harmine, and the 5-HT2 agonist DMT. Cotreatments with combinations of MAO-A inhibitors and DMT, or DYRK1A inhibitors and DMT were also examined. Neurons that were cotreated with DYRK1A inhibitors and 10 μM DMT exhibited dendritic arbor complexity comparable to those treated with 90 μM DMT. The same effect was not observed when cotreating with MAO-A inhibitors and DMT. All treatments were 10 μM unless otherwise noted. VEH=0.2% DMSO.

FIG. 12 shows Compound HTW_A_8 fumarate is selective for kinases in the CMGC family. TreeSpot™ representations of kinome scan data for HTW_A_8 fumarate and harmine at 97 kinases representing each family of the kinome. Compounds were screened at 10 μM using the DiscoverX KinomeSCAN™ assay. Results are reported as the % activity relative to the vehicle (i.e., DMSO) control, where lower values indicate higher affinity (see Table 1 for full scan data). The activity of HTW_A_8 fumarate at PIK3CG was minimal (33%) and barely met the threshold for inclusion. Compound HTW_A_8 fumarate did not exhibit activity for any other kinase in the LIPID family. Data for harmine were obtained from a previous report.

FIG. 13 shows Compound HTW_A_8 fumarate significantly increases dendritic spine density. (A) Representative images of cortical neurons (DIV15) treated with compounds (10 μM) for 24 h. (B) Quantification of dendritic spine density (n=29-55 neurons per treatment condition). Data are represented as mean±SEM. *p<0.05, **p<0.01, ***p<0.001, ****p<0.0001, as compared to the vehicle control following a one-way ANOVA with Dunnett's post hoc test. VEH=vehicle (0.2% DMSO).

FIG. 14 shows Harmine prevents pTau accumulation in cortical neurons, while isoDMT does not. Pretreatment of harmine blocks hyperphosphorylation of Tau in rat cortical cultures induced by okadaic acid treatment. Each data point represents a well average. Data are represented as mean f SEM. *p<0.05, **p<0.01, ***p<0.001, ****p<0.0001, as compared to the vehicle control following a one-way ANOVA with Dunnett's post hoc test. VEH=vehicle: OKA=30 nM okadaic acid.

FIG. 15 shows Dual ligands HTW_A_8 fumarate and HTW_B_14 fumarate produce therapeutic effects in cortical neurons relevant to Alzheimer's disease. (A) Both HTW_A_8 fumarate and HTW_B_14 fumarate significantly increase average total neurite length per neuron in embryonic rat cortical cultures (DIV=0) when treated for 72 h. (B) Pretreatment of HTW_A_8 fumarate blocks hyperphosphorylation of Tau in rat cortical cultures induced by okadaic acid treatment. Each data point represents a well average from two independent cultures. Data are represented as mean f SEM. *p<0.05, **p<0.01, ***p<0.001, ****p<0.0001, as compared to the vehicle control following a one-way ANOVA with Dunnett's post hoc test. VEH=vehicle; DON=250 nM donepezil; OKA=30 nM okadaic acid.

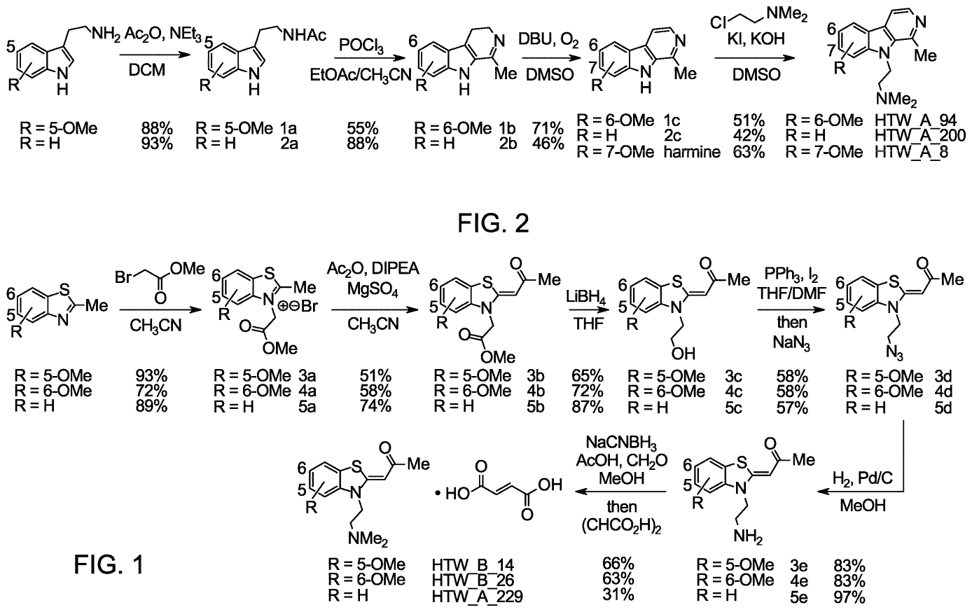

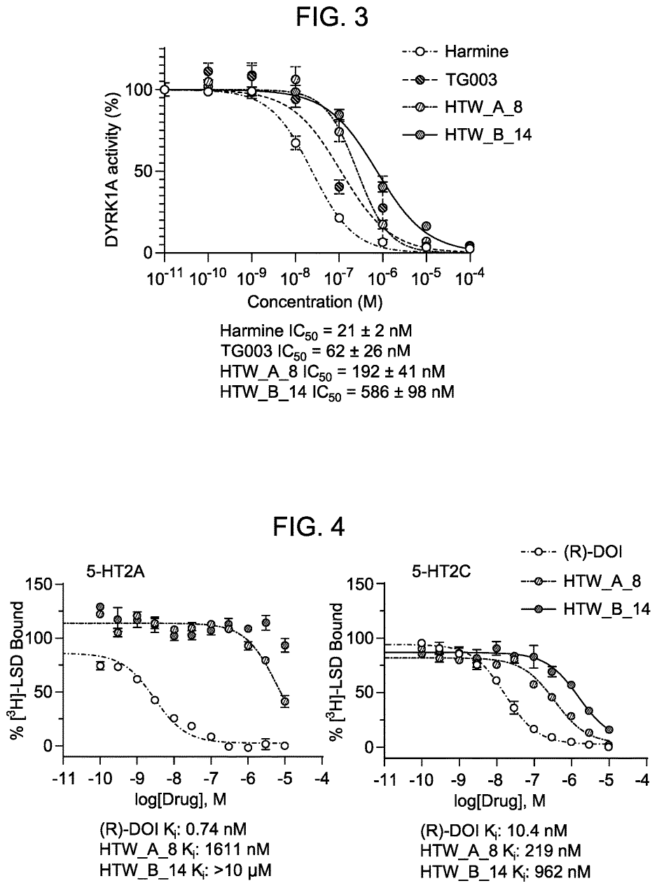

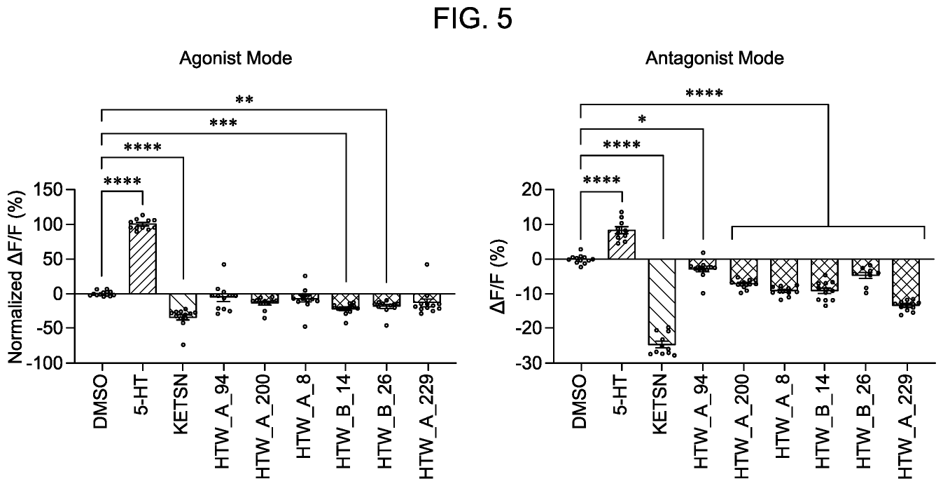

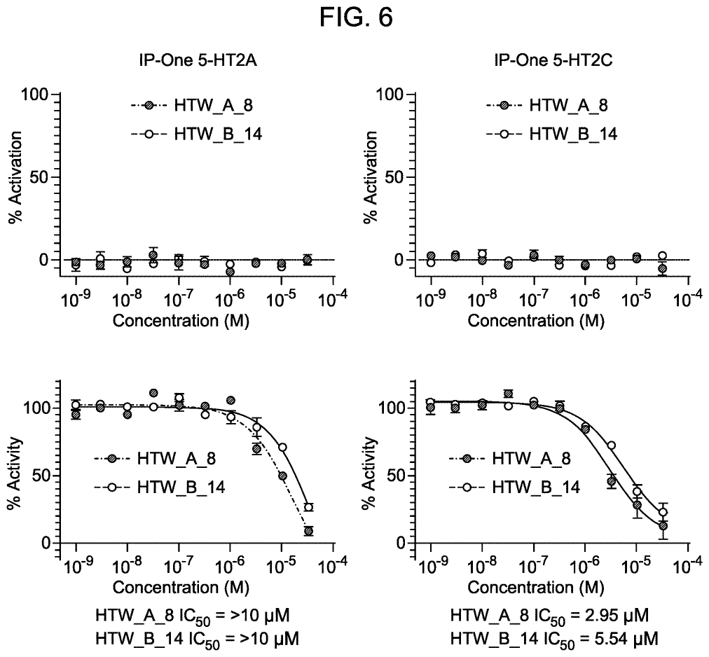

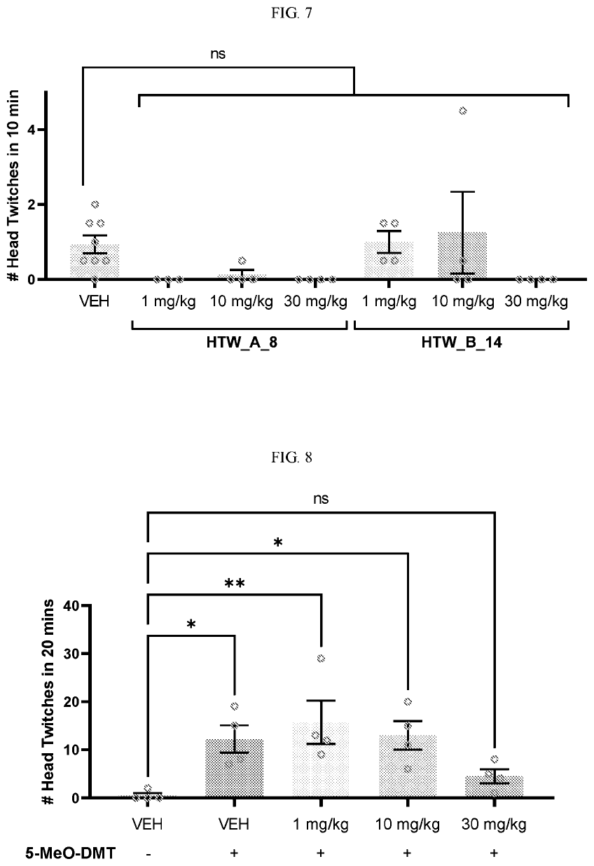

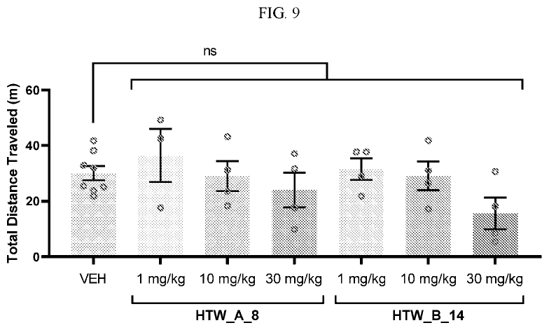

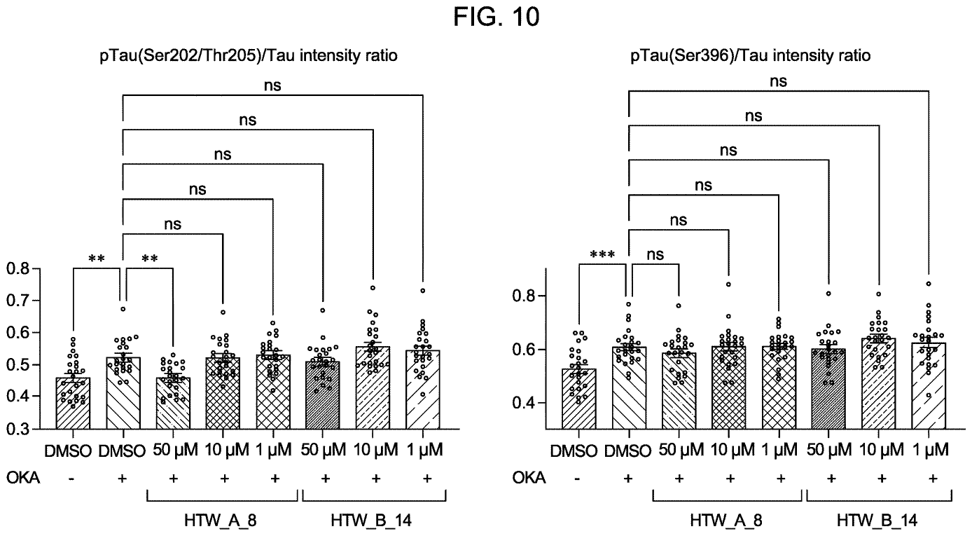

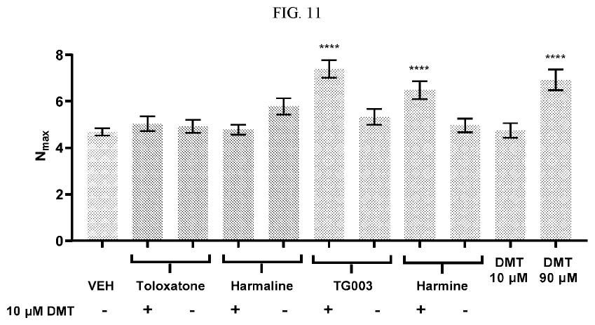

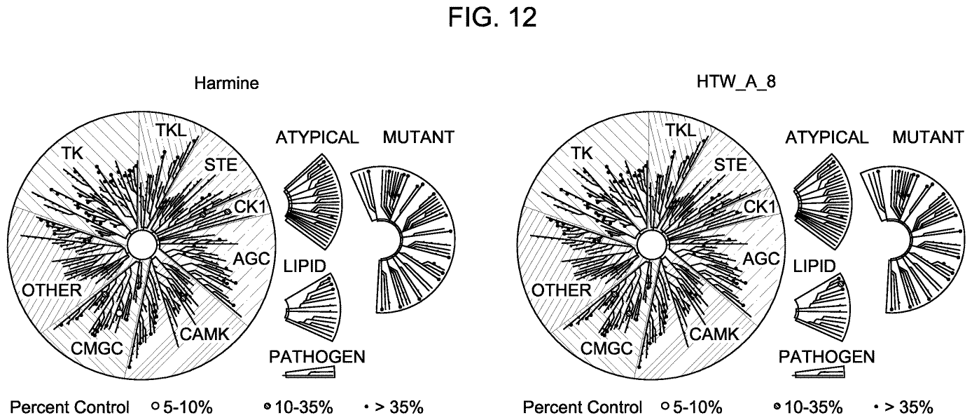

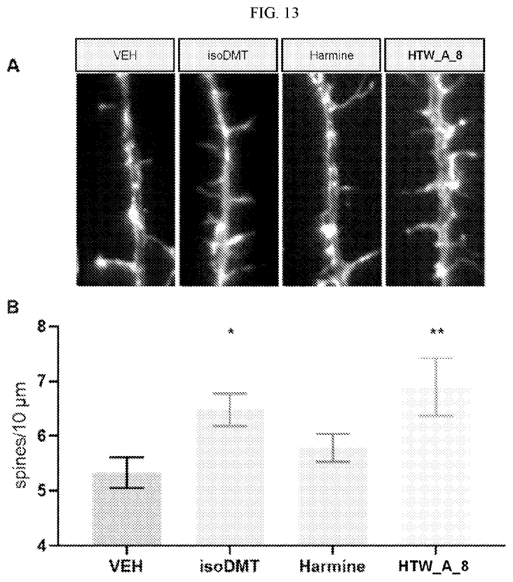

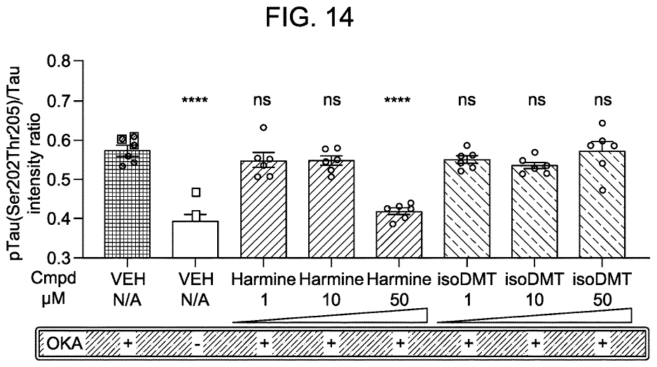

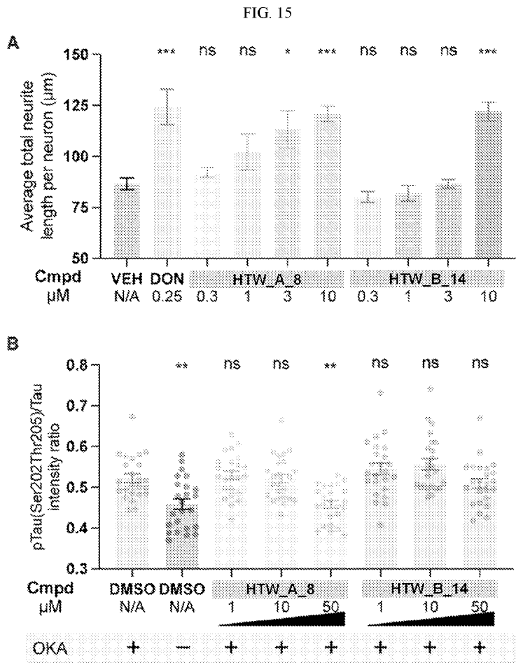

### DETAILED DESCRIPTION OF THE INVENTION

### I. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” not only include aspects with one member, but also include aspects with more than one member. For instance, the singular forms “a.” “an.” and “the” include plural referents unless the context clearly dictates otherwise. Thus, for example, reference to “a cell” includes a plurality of such cells and reference to “the agent” includes reference to one or more agents known to those skilled in the art, and so forth.

Abbreviations. VEH=vehicle; KET=ketamine; IBO=ibogaine; NOR=noribogaine; IBG=ibogainalog; TBG=tabemanthalog; KETSN=ketanserin; SI=sertindole; DOI=2,5-dimethoxy-4-iodoamphetamine; FST=forced swim test; EtOH=ethanol, DMSO=dimethyl sulfoxide, ATP=adenosine triphosphate.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂-s, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkylhydroxy” refers to an alkyl group, as defined above, where at least one of the hydrogen atoms is replaced with a hydroxy group. As for the alkyl group, alkylhydroxy groups can have any suitable number of carbon atoms, such as C₁₋₆. Exemplary alkylhydroxy groups include, but are not limited to, hydroxy-methyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Alkyl amine” refers to an alkyl group as defined within, having one or more amino groups. The amino groups can be primary, secondary or tertiary. The alkyl amine can be further substituted with a hydroxy group to form an amino-hydroxy group. Alkyl amines useful in the present invention include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present invention.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆-8, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norborane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norborene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Heterocycloalkyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Aryl” refers to an aromatic ring system having any suitable number of ring atoms and any suitable number of rings. Arv groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 5 to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₆, C₄₋₆ and C₅₋₆. In some instances, the alkyl component can be absent. The heterocycloalkyl component is as defined above. Alkyl-heterocycloalkyl groups can be substituted or unsubstituted.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within. Alkyl-heteroaryl groups can be substituted or unsubstituted.

“Psychlight” refers to Psychlight1 (SEQ ID NO:52) of WO2022/081631, and Psychlight2 (SEQ ID NO:53) of WO2022/081631, as well as sequences of WO2018/098262.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Pharmaceutically acceptable salt” refers to a compound in salt form, wherein the compound are suitable for administration to a subject. Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Isomers” refers to compounds with same chemical formula but different connectivity between the atoms in the molecule, leading to distinct chemical structures. Isomers include structural isomers and stereoisomers. Examples of structural isomers include, but are not limited to tautomers and regioisomers. Examples of stereoisomers include but are not limited to diastereomers and enantiomers.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to animals such as mammals, including, but not limited to, primates (e.g., humans), cows, sheep, goats, horses, dogs, cats, rabbits, rats, mice and the like. In certain embodiments, the subject is a human.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see. e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neural plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses. These changes can appear in neurons, glia, and in other parts of the brain. “Neuronal plasticity” refers to plasticity of the neurons.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Anxiety disorders” are defined in the Diagnostic and Statistical Manual of Mental Disorders, Fifth Edition (DSM-5 2013) as a group of disorders that share features of persistent, excessive fear and anxiety. Anxiety disorders typically have a duration of at least 6 months. In children, anxiety disorders are typically diagnosed using one or more rating scales, such as, the Pediatric Anxiety Rating Scale (PARS), the Children's Depressive Rating Scale, and the Children's Yale-Brown Obsessive Compulsive Scale. Anxiety disorders include, but are not limited to, generalized anxiety disorder, social anxiety disorder, social phobia, panic attack, panic disorder, post-traumatic stress disorder, agoraphobia, separation anxiety disorder, separation anxiety disorder, anxiety disorder induced by a substance/medication or due to another medical condition, and selective mutism, among others.

“Generalized anxiety disorder” is excessive worry about a variety of everyday problems occurring more days than not for at least 6 months. “Social anxiety disorder” is a marked fear or anxiety about one or more social situations and is interchangeable with “social phobia”. “Panic attacks” are the abrupt onset of intense fear or discomfort associated with symptoms such as heart palpitations, sweating, dizziness, or nausea. “Panic disorder” is diagnosed in patients with recurrent unexpected panic attacks. “Post-traumatic stress disorder” (or “PTSD”) is a condition that can occur in people who have been exposed to an actual or threatened death, serious injury, or sexual violence, wherein the individual experiences recurrent distressing memories, flashbacks, psychological distress, and/or physiological reactions to cues following the event. “Agoraphobia” is a condition wherein an individual has marked fear about situations such as being in public spaces, standing in crowds, or being outside of their home alone. “Separation anxiety disorder” is developmentally inappropriate and excessive fear or anxiety concerning separation from those to whom the individual is attached. “Selective mutism” is a condition characterized by consistent failure to speak in specific social situations in which there is an expectation to speak (e.g., in school), despite having the ability to speak in other situations.

“Combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

“Neurotrophic factors” or “NF” refers to a family of soluble peptides or proteins which support the survival, growth, and differentiation of developing and mature neurons. Most NFs exert their trophic effects on neurons by signaling through tyrosine kinases, usually a receptor tyrosine kinase. In the mature nervous system, they promote neuronal survival, induce synaptic plasticity, and modulate the formation of long-term memories. NFs also promote the initial growth and development of neurons in the central nervous system and peripheral nervous system. Some NFs are also released by the target tissue in order to guide the growth of developing axons. According to the invention, the term “neurotrophic factor” encompasses all neurotrophins, growth factors, and other substances that promote survival and repair of the cells of the nervous system.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A}) are modulators of the receptor.

“Agonism, “agonist” or “agonisin” refers to the activation of a receptor or enzyme by a modulator, or agonist, to produce a biological response. By way of example only, “5HT_{2A} agonist” can be used to refer to a compound that exhibits an EC₅₀ with respect to 5HT_{2A} activity of no more than about 100 μM. In some embodiments, “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism”, “antagonist” or “antagonizing” refers to the inactivation of a receptor or enzyme by a modulator, or antagonist. Antagonism of a receptor, for example, is when a molecule binds to the receptor and does not allow activity to occur. An antagonist has no activity in the absence of an agonist or inverse agonist but can block the activity of either, causing no change in the biological response.

“IC₅₀” refers to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, IC₅₀ refers to the concentration of a modulator (e.g., an antagonist or inhibitor) that is required for 50% inhibition of a receptor, for example, 5HT_{2A}.

### II. Compounds

The present invention provides N-substituted indoles and other heterocyclic compounds useful for the treatment of a variety of brain disorders and other conditions. In some embodiments, the N-substituted indoles and other heterocyclic compounds provided herein are 5-HT_{2A} modulators and promote neural plasticity (e.g., cortical structural plasticity).

In some embodiments, the present invention provides a compound of Formula I or Formula II:

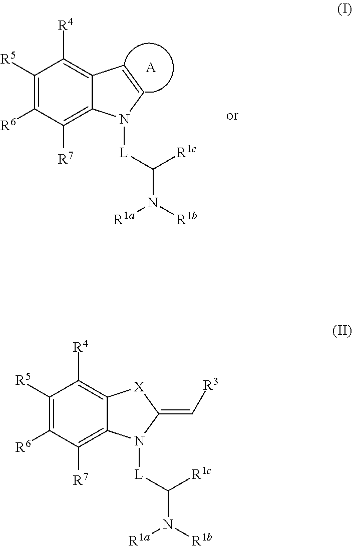

or a pharmaceutically acceptable salt thereof, wherein:
R^{1a}, R^{1b}, and R^{1c} are each independently hydrogen, C₁₋₆ alkyl, C₃₋₈ cycloalkyl, or C₄₋₁₄ alkyl-cycloalkyl;
alternatively. two of R^{1a}, R^{1b}, and R^{1c} are combined with the atoms to which they are attached to form a C₃₋₁₂ heterocycloalkyl;
Ring A is a 5 to 6 membered heteroaryl ring having 1 to 3 heteroatoms each independently N, O, or S, substituted with 0 to 3 R² groups;
R², R⁴, R⁵, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8c}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
alternatively, R⁴ and R⁵, R⁵ and R⁶, or R⁶ and R⁷ are combined with the atoms to which they are each attached to form a C₃₋₆ cycloalkyl, C₃₋₆ heterocycloalkyl.

C₆₋₁₂ aryl, or C₅₋₁₀ heteroaryl; and
R³ is C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{8a} is C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{8b}, R^{8c} and R^{8d} are each independently H or C₁₋₆ alkyl;
X is O or S; and
L is C₁₋₆ alkylene.

In some embodiments, the present invention provides a compound of Formula I:

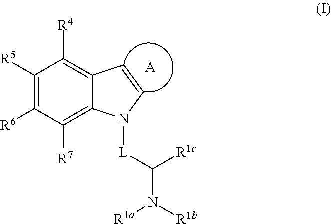

or a pharmaceutically acceptable salt thereof, wherein:
R^{1a}, R^{1b}, and R^{1c} are each independently hydrogen, C₁₋₆ alkyl, C₃₋₈ cycloalkyl, or C₄₋₁₄ alkyl-cycloalkyl;
alternatively, two of R^{1a}, R^{1b}, and R^{1c} are combined with the atoms to which they are attached to form a C₃₋₁₂ heterocycloalkyl;
Ring A is a 5 to 6 membered heteroaryl ring having 1 to 3 heteroatoms each independently N, O, or S, substituted with 0 to 3 R² groups;
R², R⁴, R⁵, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}. —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
alternatively, R⁴ and R⁵, R⁵ and R⁶, or R⁶ and R⁷ are combined with the atoms to which they are each attached to form a C₃₋₆ cycloalkyl, C₃₋₆ heterocycloalkyl, C₆₋₁₂ aryl, or C₅₋₁₀ heteroaryl; and
R^{8a} is C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl; R^{8b}, R^{8c} and R^{8d} are each independently H or C₁₋₆ alkyl; and
L is C₁₋₆ alkylene.

In some embodiments, the present invention provides a compound of Formula II:

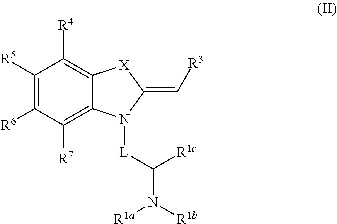

or a pharmaceutically acceptable salt thereof, wherein:
R^{1a}, R^{1b}, and R^{1c} are each independently hydrogen, C₁₋₆ alkyl, C₃₋₈ cycloalkyl, or C₄₋₁₄ alkyl-cycloalkyl;
alternatively, two of R^{1a}, R^{1b}, and R^{1c} are combined with the atoms to which they are attached to form a C₃₋₁₂ heterocycloalkyl;
R⁴, R⁵, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
alternatively, R⁴ and R⁵, R⁵ and R⁶, or R⁶ and R⁷ are combined with the atoms to which they are each attached to form a C₃₋₆ cycloalkyl, C₃₋₆ heterocycloalkyl, C₆₋₁₂ aryl, or C₅₋₁₀ heteroaryl; and
R³ is C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R⁸R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{8a} is C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{8b}, R^{8c} and R^{8d} are each independently H or C₁₋₆ alkyl;
X is O or S; and
L is C₁₋₆ alkylene.

In some embodiments, the present invention provides a compound of Formula I, or II, or a pharmaceutically acceptable salt thereof, wherein the compound has the structure of Formula Ia or Formula IIa:

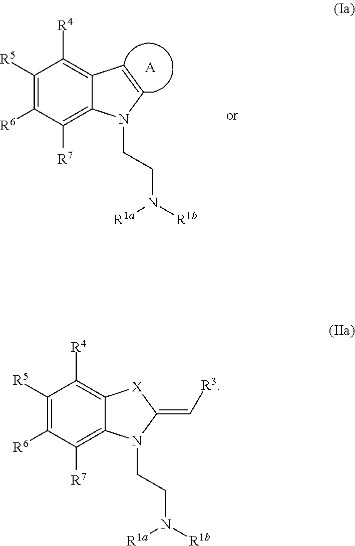

In some embodiments, the present invention provides a compound of Formula I, or a pharmaceutically acceptable salt thereof, wherein the compound has the structure of Formula Ia:

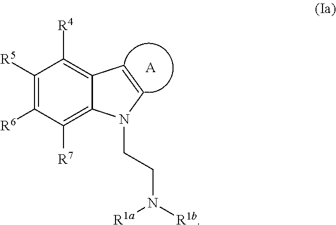

In some embodiments, the present invention provides a compound of II, or a pharmaceutically acceptable salt thereof, wherein the compound has the structure of Formula IIa:

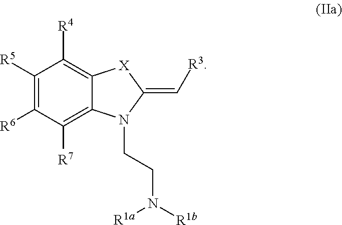

In some embodiments, the present invention provides a compound of Formula I, Ia, II, or IIa, or a pharmaceutically acceptable salt thereof, wherein the compound has the structure of Formula Ib or Formula IIb:

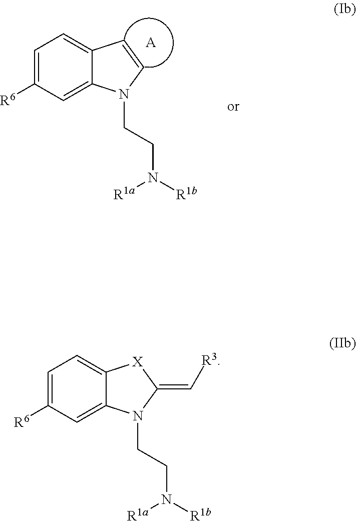

In some embodiments, the present invention provides a compound of Formula I, or Ia, or a pharmaceutically acceptable salt thereof, wherein the compound has the structure of Formula Ib:

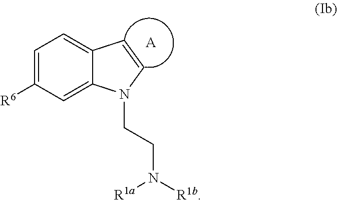

In some embodiments, the present invention provides a compound of II, or IIa, or a pharmaceutically acceptable salt thereof, wherein the compound has the structure of Formula IIb:

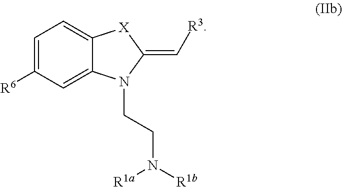

In some embodiments, the present invention provides a compound of Formula I, Ia, II, or IIa, or a pharmaceutically acceptable salt thereof, wherein the compound has the structure of Formula Ic or Formula IIc:

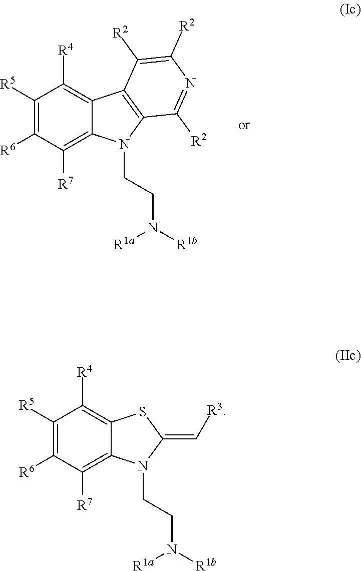

In some embodiments, the present invention provides a compound of Formula I, or Ia, or a pharmaceutically acceptable salt thereof, wherein the compound has the structure of Formula Ic:

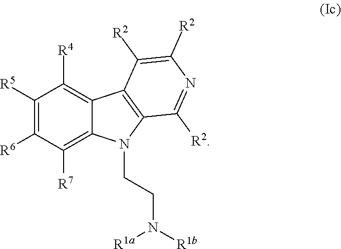

In some embodiments, the present invention provides a compound of Formula II, or IIa, or a pharmaceutically acceptable salt thereof, wherein the compound has the structure of Formula IIc:

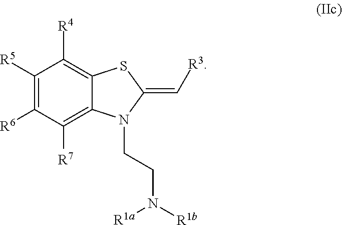

In some embodiments, the present invention provides a compound of Formula I, Ia, Ib, Ic, II, IIa, IIb. or IIc, or a pharmaceutically acceptable salt thereof, wherein the compound has the structure of Formula Ic-1 or Formula IIc-1:

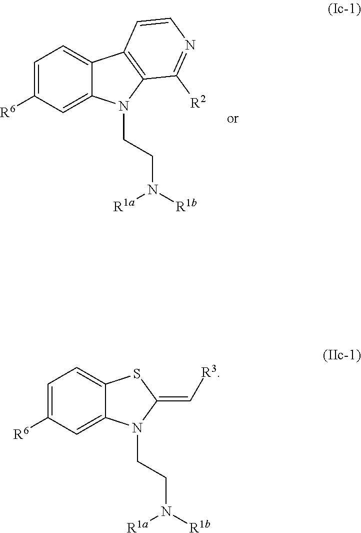

In some embodiments, the present invention provides a compound of Formula I, Ia, Ib, or Ic, or a pharmaceutically acceptable salt thereof, wherein the compound has the structure of Formula Ic-1:

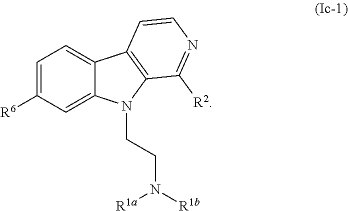

In some embodiments, the present invention provides a compound of Formula II.

IIa, IIb, or IIc, or a pharmaceutically acceptable salt thereof wherein the compound has the structure of Formula IIc-1:

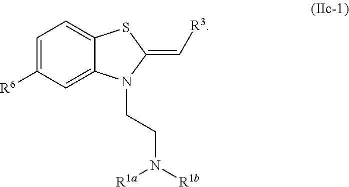

In some embodiments, the present invention provides a compound of Formula I, Ia, Ib, Ic, Ic-1. II, IIa, IIb, IIc, or IIc-1, or a pharmaceutically acceptable salt thereof, wherein R^{1a} and R^{1b} are each independently hydrogen, C₁₋₆ alkyl, C₃₋₈ cycloalkyl, or C₄₋₁₄ alkyl-cycloalkyl; and R^{1c} is hydrogen. In some embodiments, the present invention provides a compound of Formula I, Ia, Ib, Ic, Ic-1, II, IIa, IIb, IIc, or IIc-1, or a pharmaceutically acceptable salt thereof, wherein R^{1a} and R^{1b} are each independently hydrogen, or C₁₋₆ alkyl; and R^{1c} is hydrogen

In some embodiments, the present invention provides a compound of Formula I, Ia, Ib, Ic, or Ic-1, or a pharmaceutically acceptable salt thereof, wherein In some embodiments, the present invention provides a compound of Formula I, Ia, Ib, Ic, or Ic-1, or a pharmaceutically acceptable salt thereof, wherein Ring A is a 5 to 6 membered heteroaryl ring having 1 to 3 heteroatoms each independently N, substituted with 0 to 3 R² groups. In some embodiments, the present invention provides a compound of Formula I, Ia, Ib, Ic, or Ic-1, or a pharmaceutically acceptable salt thereof, wherein Ring A is a 6 membered heteroaryl ring having 1 to 3 heteroatoms each independently N, substituted with 0 to 3 R² groups. In some embodiments, the present invention provides a compound of Formula I, Ia. Ib, Ic, or Ic-1, or a pharmaceutically acceptable salt thereof, wherein Ring A is a 6 membered heteroaryl ring having 1 heteroatom of N, substituted with 1 R² groups. In some embodiments, the present invention provides a compound of Formula I, Ia, Ib, Ic, or Ic-1, or a pharmaceutically acceptable salt thereof, wherein Ring A is pyridine, substituted with 1 methyl.

In some embodiments, the present invention provides a compound of Formula I, Ia, Ib, Ic, Ic-1, II, IIa, IIb, IIc, or IIc-1, or a pharmaceutically acceptable salt thereof, wherein R⁴, R⁵, R⁶ and R⁷ are each independently hydrogen, C₁₋₆ alkyl, or C₁₋₆ alkoxy. In some embodiments, the present invention provides a compound of Formula I, Ia, Ib, Ic, Ic-1, II, IIa, IIb, IIc, or IIc-1, or a pharmaceutically acceptable salt thereof, wherein R⁴, R⁵, R⁶ and R⁷ are each independently hydrogen, or C₁₋₆ alkoxy. In some embodiments, the present invention provides a compound of Formula I, Ia. Ib, Ic, Ic-1, II, IIa, IIb, IIc, or IIc-1, or a pharmaceutically acceptable salt thereof, wherein R⁴, R⁵, R⁶ and R⁷ are each independently hydrogen, or methoxy.

In some embodiments, the present invention provides a compound of Formula II, IIa, IIb, IIc, or IIc-1, or a pharmaceutically acceptable salt thereof, wherein In some embodiments, the present invention provides a compound of Formula II, IIa, IIb, IIc, or IIc-1, or a pharmaceutically acceptable salt thereof, wherein R³ is —C(O)R^{8b}, —C(O)OR^{8b}, or —C(O)N(R^{8b}R^{8c}); and R^{8b} and R^{8c} are each independently H or C₁₋₆ alkyl. In some embodiments, the present invention provides a compound of Formula II, IIa, IIb, IIc, or IIc-1, or a pharmaceutically acceptable salt thereof, wherein R³ is —C(O)R^{8b}; and R^{8b} is C₁₋₆ alkyl. In some embodiments, the present invention provides a compound of Formula II, IIa, IIb, IIc, or IIc-1, or a pharmaceutically acceptable salt thereof, wherein R³ is —C(O)Me.

In some embodiments, the present invention provides a compound of Formula II, IIa, IIb, IIc, or IIc-1, or a pharmaceutically acceptable salt thereof, wherein X is O or S. In some embodiments, the present invention provides a compound of Formula II, IIa, IIb, IIc, or IIc-1, or a pharmaceutically acceptable salt thereof, wherein X is S.

In some embodiments, the present invention provides a compound of Formula I, Ia, Ib, Ic, Ic-1. II, IIa, IIb, IIc. or IIc-1, or a pharmaceutically acceptable salt thereof, wherein L is C₁₋₃ alkylene. In some embodiments, the present invention provides a compound of Formula I, Ia, Ib, Ic, Ic-1, II, IIa, IIb, IIc, or IIc-1, or a pharmaceutically acceptable salt thereof, wherein L is C₂ alkylene.

In some embodiments, the present invention provides a compound of Formula I, Ia, Ib, Ic, Ic-1, II, IIa, IIb, IIc, or IIc-1, or a pharmaceutically acceptable salt thereof, wherein R^{1a} and R^{1b} are each C₁₋₆ alkyl; Ring A is a 6 membered heteroaryl ring having 1 heteroatom of N, substituted with 1 R² groups; R² is C₁₋₆ alkyl; R⁴, R⁵, R⁶ and R⁷ are each independently hydrogen, C₁₋₆ alkyl, or C₁₋₆ alkoxy; R³ is —C(O)R^{8b}, —C(O)OR^{8b}, or —C(O)N(R^{8b}R^{8c}); R^{8b} and R^{8c} are each C₁₋₆ alkyl; and X is O or S.

In some embodiments, the present invention provides a compound of Formula I, La, Ib, Ic, Ic-1, II, IIa, IIb, IIc, or IIc-1, or a pharmaceutically acceptable salt thereof, having the structure of:

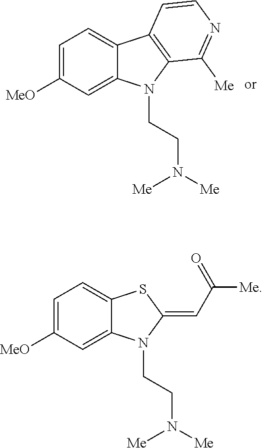 [SMILES: COc1ccc2c3ccnc(C)c3n(CCN(C)C)c2c1 ; "or" ; COc1ccc2c(c1)N(CCN(C)C)/C(=C/C(C)=O)S2]

In some embodiments, the present invention provides a compound of Formula I, La, Ib, Ic, or Ic-1, or a pharmaceutically acceptable salt thereof, having the structure of:

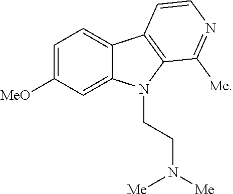 [SMILES: COc1ccc2c3ccnc(C)c3n(CCN(C)C)c2c1]

In some embodiments, the present invention provides a compound of Formula I, La, Ib, Ic, or Ic-1, having the structure of:

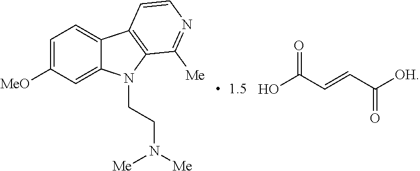 [SMILES: COc1ccc2c3ccnc(C)c3n(CCN(C)C)c2c1 ; O=C(O)/C=C/C(=O)O]

In some embodiments, the present invention provides a compound of Formula II, IIa, IIb, IIc, or IIc-1, or a pharmaceutically acceptable salt thereof, having the structure of:

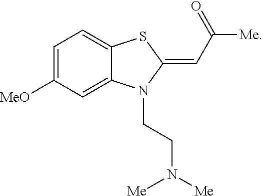 [SMILES: COc1ccc2c(c1)N(CCN(C)C)/C(=C/C(C)=O)S2]

In some embodiments, the present invention provides a compound of Formula II, IIa, IIb, IIc, or IIc-1, having the structure of.

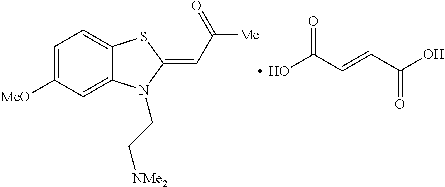 [SMILES: COc1ccc2c(c1)N(CCN(C)C)/C(=C/C(C)=O)S2 ; O=C(O)/C=C/C(=O)O]

The compounds of the present invention can also be in the salt forms, such as acid or base salts of the compounds of the present invention. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The present invention also includes isotopically-labeled compounds of the present invention, wherein one or more atoms are replaced by one or more atoms having specific atomic mass or mass numbers. Examples of isotopes that can be incorporated into compounds of the invention include, but are not limited to, isotopes of hydrogen, carbon, nitrogen, oxygen, fluorine, sulfur, and chlorine (such as ²H, ³H, ¹³C, ¹⁴C, ¹⁵N, ¹⁸O, ¹⁷O, ¹⁸F, ³⁵S and ³⁶Cl). Isotopically-labeled compounds of the present invention are useful in assays of the tissue distribution of the compounds and their prodrugs and metabolites; preferred isotopes for such assays include ³H and ¹⁴C. In addition, in certain circumstances substitution with heavier isotopes, such as deuterium (²H), can provide increased metabolic stability, which offers therapeutic advantages such as increased in vivo half-life or reduced dosage requirements. Isotopically-labeled compounds of this invention can generally be prepared according to the methods known by one of skill in the art by substituting an isotopically-labeled reagent for a non-isotopically labeled reagent. Compounds of the present invention can be isotopically labeled at positions adjacent to the basic amine, in aromatic rings, and the methyl groups of methoxy substituents.

The present invention includes all tautomers and stereoisomers of compounds of the present invention, either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers are within the scope of the present invention. Compounds according to the present invention can be prepared using diastereomers, enantiomers or racemic mixtures as starting materials. Furthermore, diastereomer and enantiomer products can be separated by chromatography, fractional crystallization or other methods known to those of skill in the art.

In some embodiments, a compound provided herein, including pharmaceutically acceptable salts and solvates thereof, is a non-hallucinogenic psychoplastogen.

### III. Pharmaceutical Compositions and Formulations

In some embodiments, provided herein is a pharmaceutical composition, comprising a therapeutically effective amount of a compound of the present invention, or a pharmaceutically acceptable salt thereof.

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences. Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compound the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compound of the present invention mixed with a filler or binders such as lactose or starches, lubricants such as talc or magnesium stearate, and, optionally, stabilizers. In soft capsules, the compound of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of the present invention is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J Hosp. Pharm. 46:1576-1587, 1989).

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds of the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods of the present invention.

### IV. Methods of Treatment

The compounds of the present invention can be used for increasing neural and neuronal plasticity. The compounds of the present invention can also be used to treat any brain disease. The compounds of the present invention can also be used for increasing at least one of translation, transcription or secretion of neurotrophic factors.

In some embodiments, a compound of the present invention is used to treat a brain disorder. In some embodiments, the compounds have, for example, anti-addictive properties, antidepressant properties, anxiolytic properties, or a combination thereof. In some embodiments, the brain disorder is a neuropsychiatric disease. In some embodiments, the brain disorder is a mood or anxiety disorder. In some embodiments, the brain disorder is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the brain disorder is a migraine or cluster headache. In some embodiments, the brain disorder is a neurodegenerative disorder, Alzheimer's disease, or Parkinson's disease. In some embodiments, the brain disorder is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the brain disorder is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the brain disorder is post-traumatic stress disorder (PTSD), addiction (e.g., substance use disorder), schizophrenia, depression, or anxiety. In some embodiments, the brain disorder is addiction (e.g., substance use disorder). In some embodiments, the brain disorder is depression. In some embodiments, the brain disorder is anxiety. In some embodiments, the brain disorder is post-traumatic stress disorder (PTSD). In some embodiments, the brain disorder is stroke or traumatic brain injury. In some embodiments, the brain disorder is schizophrenia.

In some embodiments, a compound of the present invention is used for increasing neural plasticity. In some embodiments, a compound of the present invention is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the compounds of the present invention have activity as 5-HT2 modulators. In some embodiments, the compounds of the present invention have activity as 5-HT2 modulators. In some embodiments, the compounds of the present invention elicit a biological response by activating a 5-HT2 receptor (e.g., allosteric modulation or modulation of a biological target that activates a 5-HT2 receptor). 5-HT2 modulation has been correlated with the promotion of neural plasticity (Ly et al., 2018). In some embodiments, hallucinogenic potential of a compound of the present invention is determined in vitro. In some embodiments, hallucinogenic potential of a compound of the present invention is determined using a 5-HT2 sensor assay. In some embodiments, the 5-HT2 sensor assay is in an agonist mode or an antagonist mode. In some embodiments, the 5-HT2 sensor assay is in an agonist mode. In some embodiments, a compound of the present invention that does not activate the sensor in agonist mode has non-hallucinogenic potential. In some embodiments, a compound of the present invention that does not activate the sensor in agonist mode is a non-hallucinogenic compound.

In some embodiments, the compounds described herein are 5-HT2 (5-HT_{2A} and/or 5-HT_{2c}) modulators. In some embodiments, the compounds described herein are 5-HT2 modulators and promote neural plasticity (e.g., cortical structural plasticity). In some embodiments, the compounds described herein are 5-HT2 modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT2 modulators (e.g., 5-HT2 agonists and 5-HT2 antagonists) are non-hallucinogenic. In some embodiments, non-hallucinogenic 5-HT2 modulators (e.g., 5-HT2 agonists and 5-HT2 antagonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, non-hallucinogenic 5-HT2 modulators (e.g., 5-HT2 agonists and 5-HT2 antagonists) are used to treat neurological diseases. In some embodiments, the neurological diseases comprise decreased neural plasticity, decreased cortical structural plasticity, decreased 5-HT2 receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT2 modulators (e.g., 5-HT2 agonists and 5-HT2 antagonists) are used for increasing neural plasticity. In some embodiments, non-hallucinogenic 5-HT2 modulators (e.g., 5-HT2 agonists and 5-HT2 antagonists) are used for increasing neuronal plasticity. In some embodiments, non-hallucinogenic 5-HT2 modulators (e.g., 5-HT2 agonists and 5-HT2 antagonists) are used for treating a brain disorder. In some embodiments, non-hallucinogenic 5-HT2 modulators (e.g., 5-HT2 agonists and 5-HT2 antagonists) are used for increasing at least one of translation, transcription, or secretion of neurotrophic factors.

### A. Methods for Increasing Neural and Neuronal Plasticity

Neural plasticity refers to the ability of the brain to change structure and/or function throughout a subject's life. Increasing neural plasticity can include increasing neuronal plasticity. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neural plasticity can treat a brain disorder, a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, increasing neuronal plasticity can treat a brain disorder, a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present invention provides a method for increasing neural plasticity, comprising contacting a neural cell with a compound of the present invention, or a pharmaceutically acceptable salt thereof, in an amount sufficient to increase neural plasticity of the neuronal cell. In some embodiments, increasing neural plasticity improves a brain disorder described herein.

In some embodiments, the present invention provides a method for increasing neuronal plasticity, comprising contacting a neuronal cell with a compound of the present invention, or a pharmaceutically acceptable salt thereof, in an amount sufficient to increase neuronal plasticity of the neuronal cell. In some embodiments, increasing neuronal plasticity improves a brain disorder described herein.

In some embodiments, a compound of the present invention is used to increase neural plasticity. In some embodiments, the compounds used to increase neural plasticity have, for example, anti-addictive properties, antidepressant properties, anxiolytic properties, or a combination thereof. In some embodiments, decreased neural plasticity is associated with a neuropsychiatric disease. In some embodiments, a compound of the present invention is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neural plasticity of any compound of the present invention is a phenotypic assay, a dendritogenesis assay, a spinogenesis assay, a synaptogenesis assay, a Sholl analysis, a concentration-response experiment, a 5-HT2 agonist assay, a 5-HT2 antagonist assay, a 5-HT binding assay, or a 5-HT2 blocking experiment (e.g., ketanserin blocking experiments).

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT2 agonist assay, a 5-HT2 antagonist assay, a 5-HT binding assay, or a 5-HT2 blocking experiment (e.g., ketanserin blocking experiments). In some embodiments, the experiment or assay to determine the hallucinogenic potential of any compound of Formula I or Formula (Ia) is a mouse head-twitch response (HTR) assay.

In some embodiments, the present invention provides a method for increasing neural plasticity, comprising contacting a neural cell with a compound of the present invention.

In some embodiments, the present invention provides a method for increasing neuronal plasticity, comprising contacting a neuronal cell with a compound of the present invention.

### B. Methods of Treating a Brain Disorder

In some embodiments, the present invention provides a method of treating a disease, including administering to a subject in need thereof, a therapeutically effective amount of a compound of the present invention. In some embodiments, the present invention provides a method of treating a brain disorder, including administering to a subject in need thereof, a therapeutically effective amount of a compound of the present invention. In some embodiments, the present invention provides a method of treating a brain disorder with combination therapy, including administering to a subject in need thereof, a therapeutically effective amount of a compound of the present invention and at least one additional therapeutic agent.

In some embodiments, the present invention provides a method of treating a brain disorder, comprising administering to a subject in need thereof a therapeutically effective amount of a compound of Formula I or Formula II of the present invention.

In some embodiments, the present invention provides a method of treating a brain disorder, comprising administering to a subject in need thereof a therapeutically effective amount of a DYRK1A modulator and a 5-HT2 modulator, wherein each modulator is present in a sub-therapeutic amount.

In some embodiments, the present invention provides a method of treating a brain disorder, comprising administering to a subject in need thereof a therapeutically effective amount of a compound having a DYRK1A binding moiety and a 5-HT binding moiety.

In some embodiments, 5-HT2 modulators (e.g., 5-HT2 agonists and 5-HT2 antagonists) are used to treat a brain disorder. In some embodiments, the brain disorders comprise decreased neural plasticity, decreased cortical structural plasticity, decreased 5-HT2 receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present invention is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, schizophrenia, and addiction (e.g., substance abuse disorder). In some embodiments, brain disorders include, for example, migraines, addiction (e.g., substance use disorder), depression, and anxiety.

In some embodiments, the brain disorder is one or more of generalized anxiety disorder, phobia, social anxiety disorder, social phobia, panic disorder, panic attack, post-traumatic stress disorders, separation anxiety disorder, selective mutism, agoraphobia, or an anxiety disorder induced by a substance/medication or due to another medical condition. In some embodiments, the methods reduce at least one symptom of anxiety disorder in the subject, such as fear, anxiety, separation anxiety, and/or panic attacks (e.g. frequency and severity of attacks). In some embodiments, the brain disorder is social anxiety disorder. In some embodiments, the brain disorder is post-traumatic stress disorder.

In some embodiments, the present invention provides a method of treating a brain disorder, comprising administering to a subject in need thereof a therapeutically effective amount of a compound of the present invention, or a pharmaceutically acceptable salt thereof, thereby treating the brain disorder.

In some embodiments, the brain disorder is a neurodegenerative disorder.

Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, Down's syndrome or substance use disorder.

In some embodiments, the brain disorder is a neurodegenerative disorder. Alzheimer's, or Parkinson's disease. In some embodiments, the brain disorder is Down's syndrome. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder. In some embodiments, the brain disorder is stroke or traumatic brain injury.

In some embodiments, the method further comprises administering one or more additional therapeutic agent that is lithium, Olanzapine (Zyprexa), Quetiapine (Seroquel), Risperidone (Risperdal), Ariprazole (Abilify), Ziprasidone (Geodon), Clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Pamate), diazepam (Valium), alprazolam (Xanax), or clonazepam (Klonopin).

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Non-limiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline.

In some embodiments, the present invention provides a compound of the present invention, or a pharmaceutically acceptable salt thereof, or a composition of the present invention, for use in treating a brain disorder.

In some embodiments, the present invention provides use of a compound of the present invention, or a pharmaceutically acceptable salt thereof, or a composition of the present invention, for the manufacture of a medicament for use in treating a brain disorder.

### C. Methods of Increasing at Least One of Translation, Transcription, or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neural plasticity, neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increase neural plasticity. In some embodiments, increasing at least one of translation, transcription, or secretion of neurotrophic factors can increase neuronal plasticity. In some embodiments, increasing at least one of translation, transcription, or secretion of neurotrophic factors can include promoting neuronal growth, promoting neuritogenesis, promoting synaptogenesis, promoting dendritogenesis, increasing dendritic arbor complexity, and/or increasing dendritic spine density.

In some embodiments, the present invention provides a method for increasing at least one of translation, transcription or secretion of neurotrophic factors, comprising contacting a neural cell with a compound of the present invention, or a pharmaceutically acceptable salt thereof, in an amount sufficient to increase neural plasticity of the neuronal cell.

In some embodiments, the present invention provides a method for increasing at least one of translation, transcription or secretion of neurotrophic factors, comprising contacting a neuronal cell with a compound of the present invention, or a pharmaceutically acceptable salt thereof, in an amount sufficient to increase neuronal plasticity of the neuronal cell.

In some embodiments, 5-HT2 modulators (e.g., 5-HT2 agonists and 5-HT2 antagonists) are used to increase at least one of translation, transcription, or secretion of neurotrophic factors. In some embodiments, a compound of Formula I or Formula (Ia) described herein is used increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

The neurotrophic factor can be any member of the neurotrophin family such as, but not limited to, Nerve growth factor (NGF), Brain-derived neurotrophic factor (BDNF), neurotrophin 3 (NT-3), neurotrophin 4/5 (NT-4/5), neurotrophin 6 (NT-6), or neurotrophin 7 (NT-7); the neuropoietic cytokines or neurokines such as members of the Ciliary neurotrophic factor (CNTF) family, Leukemia inhibitory factor (LIF), cholinergic differentiation factor, cardiotrophin-1, oncostatin M, growth promoter activity factor, tumor necrosis factor (TNF), interleukin-6 (IL-6), prolactin, growth hormone (GH), leptin, interferon-.alpha., interferon-.beta., and interferon-.gamma.; Epidermal Growth factor (EGF) and Transforming Growth Factor (TGF) families such as of EGF, p185erbB2, p160erbB3, p180erbB4, neuregulin family including neu differentiation factor or heregulin, acetylcholine receptor-inducing activity, and Glial growth factors (GGFs), TGFalpha, TGFbeta, Glial cell line-derived neurotrophic factor (GDNF), artemin, neurturin, persephin, osteogenic protein-1 (OP-1), bone morphogenetic proteins (BMPs), and growth differentiation factors; Fibroblast growth factor (FGF) family; Insulin-like growth factor (IGF) family; platelet-derived growth factor (PDGF) family; Hepatocyte growth factor (HGF) family; granulocyte-colony stimulating factor (G-CSF) family; neuroimmunophilins; pigment epithelium-derived factor (PEDF) family; activity-dependent neurotrophic factors such as activity-dependent neuroprotective protein (ADNP) and neuritin (activity-induced neurotrophic factor); angiogenesis growth factor family; vascular endothelial growth factor (VEGF) family; cerebral dopamine neurotrophic factor (CDNF) family; mesencephalic astrocyte-derived neurotrophic factor (MANF) family; Ephrins family such as ephrin A1, ephrin A2, ephrin A3, ephrin A4, ephrin A5, ephrin B1, ephrin B2, and ephrin B3; DHEA; glia maturation factor; insulin; pituitary adenylate cyclase-activating peptide (PACAP); interleukin-1 (IL-1); interleukin-2 (IL-2); interleukin-3 (IL-3); interleukin-5 (IL-5); interleukin-8 (IL-8); macrophage colony-stimulating factor (M-CSF); granulocyte-macrophage colony-stimulating factor (GM-CSF); neurotactin; neurotransmitters and neuromodulators; serine protease inhibitors such as protease nexin-1, hedgehog family of inducing proteins; proteins involved in synapse formation such as agrin, laminin 2, and ARIA (ACh-inducing activity); variants and combinations thereof. In some embodiments, the neurotrophin factors can be Nerve growth factor (NGF), Brain-derived neurotrophic factor (BDNF), neurotrophin 3 (NT-3), neurotrophin 4/5 (NT-4/5), neurotrophin 6 (NT-6), neurotrophin 7 (NT-7), variants and combination thereof.

In some embodiments, the present invention provides a method for increasing at least one of translation, transcription or secretion of neurotrophic factors, comprising contacting a neural cell with a compound of Formula I or Formula IL.

In some embodiments, the present invention provides a method for increasing at least one of translation, transcription or secretion of neurotrophic factors, comprising contacting a neuronal cell with a compound of Formula I or Formula II.

### D. Methods for Modulating a 5-HT2 Receptor

In some embodiments, the present invention provides a method for modulating a 5-HT2 receptor, comprising contacting a neural cell with a compound of the present invention, or a pharmaceutically acceptable salt thereof, or a composition of the present invention, in an amount sufficient to modulate the 5-HT2 receptor.

In some embodiments, the present invention provides a method for modulating a 5-HT2 receptor, comprising contacting a neuronal cell with a compound of the present invention, or a pharmaceutically acceptable salt thereof, or a composition of the present invention, in an amount sufficient to modulate the 5-HT2 receptor.

Modulating the 5-HT2 receptor can include agonising or antagonizing the 5-HT2 receptor. In some embodiments, the present invention provides a method for agonising a 5-HT2 receptor, comprising contacting a neural cell with a compound of the present invention, or a pharmaceutically acceptable salt thereof, or a composition of the present invention, in an amount sufficient to agonise the 5-HT2 receptor. In some embodiments, the present invention provides a method for agonising a 5-HT2 receptor, comprising contacting a neuronal cell with a compound of the present invention, or a pharmaceutically acceptable salt thereof, or a composition of the present invention, in an amount sufficient to agonise the 5-HT2 receptor. In some embodiments, the present invention provides a method for antagonizing a 5-HT2 receptor, comprising contacting a neural cell with a compound of the present invention, or a pharmaceutically acceptable salt thereof, or a composition of the present invention, in an amount sufficient to antagonize the 5-HT2 receptor. In some embodiments, the present invention provides a method for antagonizing a 5-HT2 receptor, comprising contacting a neuronal cell with a compound of the present invention, or a pharmaceutically acceptable salt thereof, or a composition of the present invention, in an amount sufficient to antagonize the 5-HT2 receptor.

The 5-HT2 receptor includes, but is not limited to, the 5-HT2A, 5-HT2B, or 5-HT2C receptor. In some embodiments, The 5-HT2 receptor is at least one of 5-HT2A, 5-HT2B. or 5-HT2C receptor. In some embodiments, the 5-HT2 receptor is 5-HT2A. In some embodiments, the 5-HT2 receptor is 5-HT2B. In some embodiments, the 5-HT2 receptor is 5-HT2C.

In some embodiments, the present invention provides a method for treating a disease or condition responsive to the modulation of a 5-HT2 receptor, comprising contacting a neural cell with a compound of the present invention, or a pharmaceutically acceptable salt thereof, or a composition of the present invention, in an amount sufficient to treat the disease or condition responsive to modulation of the 5-HT2 receptor. In some embodiments, the present invention provides a method for treating a disease or condition responsive to the modulation of a 5-HT2 receptor, comprising contacting a neuronal cell with a compound of the present invention, or a pharmaceutically acceptable salt thereof, or a composition of the present invention, in an amount sufficient to treat the disease or condition responsive to modulation of the 5-HT2 receptor. The 5-HT2 receptor includes, but is not limited to, the 5-HT2A, 5-HT2B, or 5-HT2C receptor. In some embodiments. The 5-HT2 receptor is at least one of 5-HT2A, 5-HT2B, or 5-HT2C receptor. In some embodiments, the 5-HT2 receptor is 5-HT2A. In some embodiments, the 5-HT2 receptor is 5-HT2B. In some embodiments, the 5-HT2 receptor is 5-HT2C.

### V. Examples

### Materials and Methods

All reagents were obtained from commercial sources and reactions were performed using oven-dried glassware (120° C.) under an inert N₂ atmosphere unless otherwise noted. Air- and moisture-sensitive liquids and solutions were transferred via syringe or stainless-steel cannula. Organic solutions were concentrated under reduced pressure (~5 Torr) by rotary evaporation. Solvents were purified by passage under 12 psi Ar through activated alumina columns. Chromatography was performed using Fisher Chemical™ Silica Gel Sorbent (230-400 Mesh, Grade 60). Compounds purified by chromatography were typically applied to the adsorbent bed using the indicated solvent conditions with a minimum amount of added dichloromethane as needed for solubility. Thin layer chromatography (TLC) was performed on Merck silica gel 60 F254 plates (250 m). Visualization of the developed chromatogram was accomplished by fluorescence quenching or by staining with aqueous potassium permanganate or Ehrlich's reagent.

Nuclear magnetic resonance (NMR) spectra were acquired on a Bruker 400 operating at 400 and 100 MHz for ¹H and ¹³C. respectively, and are referenced internally according to residual solvent signals. Data for ¹H NMR are recorded as follows: chemical shift (6, ppm), multiplicity (s, singlet; d, doublet; t, triplet; q, quartet; quint, quintet; m, multiplet), coupling constant (Hz), and integration. Data for ¹³C NMR are reported in terms of chemical shift (6, ppm). Infrared spectra were recorded using a Thermo Nicolet iS10 Fourier transform infrared (FT-IR) spectrometer with a Smart iTX Accessory [diamond attenuated total reflection (ATR)] and are reported in the frequency of absorption (v, cm⁻¹). Liquid chromatography-mass spectrometry (LC-MS) was performed using a Waters LC-MS with an ACQUITY Arc QDa detector.

Chemistry (General). All reagents were obtained commercially unless otherwise noted. Reactions were performed using glassware that was oven dried (120° C.) unless otherwise stated. Air- and moisture-sensitive liquids and solutions were transferred via syringe or stainless-steel cannula. Organic solutions were concentrated under reduced pressure (~5 Torr) by rotary evaporation. Solvents were purified by passage under 12 psi N₂ through activated alumina columns. Chromatography was performed using Fisher Chemical™ Silica Gel Sorbent (230-400 Mesh, Grade 60). Compounds purified by chromatography were typically applied to the adsorbent bed using the indicated solvent conditions with a minimum amount of added dichloromethane as needed for solubility. Thin layer chromatography (TLC) was performed on Merck silica gel 60 F254 plates (250 m). Visualization of the developed chromatogram was accomplished by fluorescence quenching or by staining with butanolic ninhydrin or aqueous potassium permanganate.

Nuclear magnetic resonance (NMR) spectra were acquired on either a Bruker 400 operating at 400 and 100 MHz or a Varian-600 operating at 600 and 150 MHz for ¹H and ¹³C, respectively, and are referenced internally according to residual solvent signals. Data for ¹H NMR are recorded as follows: chemical shift (6, ppm), multiplicity (s, singlet; br s, broad singlet; d, doublet; t, triplet; q, quartet; quint, quintet; sext, sextet; m, multiplet), integration, and coupling constant (Hz). Data for ¹³C NMR are reported in terms of chemical shift (6, ppm). Infrared spectra were recorded using a Thermo Scientific Nicolet iS10 spectrometer with Smart iTX Accessory (diamond ATR) and are reported in frequency of absorption. Low-resolution mass spectra were obtained using a Waters LC-MS with an ACQUITY Arc QDa detector. For cellular assays, all compounds were dissolved in DMSO and stored as 10 mM stock solutions in the dark at −20° C.

All compounds tested in cellular assays were confirmed to be of >95% purity based on UHPLC analysis (Waters ACQUITY Arc) measuring absorbance at 254 and 280 nm. Mobile phase A consisted of 0.01% formic acid in water, and mobile phase B consisted of 0.01% formic acid in acetonitrile. All samples were injected at a volume of 5 μL, and the column temperature was maintained at 40° C. The method utilized a CORTECS C18, 2.7 μm, 4.6×50 mm² column, a flow rate of 0.6 mL/min. and a gradient from 10 to 90% mobile phase B over 3 min, which was maintained for an additional 2 min. As all compounds reported in this study were isolated as the fumarate salts, peaks in UHPLC traces corresponding to fumaric acid were not included in the calculation of purity.

### A. Compounds

### Example 1: 2-(6-methoxy-1-methyl-9H-pyrido[3,4-b]indol-9-yl)-N,N-dimethylethan-1-amine (HTW_A_94)

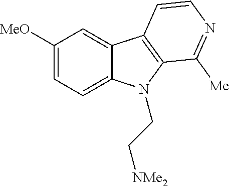 [SMILES: COc1ccc2c(c1)c1ccnc(C)c1n2CCN(C)C]

### N-(2-(5-methoxy-1H-indol-3-yl)ethyl)acetamide (1a)

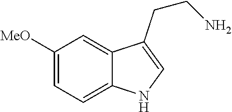 [SMILES: COc1ccc2[nH]cc(CCN)c2c1]

To a suspension of 5-MeO-tryptamine (0.150 g, 0.79 mmol, 1.0 equiv) in DCM (2.4 mL, 0.3 M) was added acetic anhydride (0.13 mL, 1.3 mmol, 1.7 equiv) and triethylamine (0.22 mL, 1.6 mmol, 2.0 equiv). The solution was stirred for 30 minutes at ambient temperature. Saturated NaHCO_{3(aq.)} (5 mL) was then added, the organic layer was collected, and the aqueous layer was extracted with DCM (3×5 mL). The combined organic extracts were washed with brine (5 mL), dried over Na₂SO₄, filtered, and concentrated under reduced pressure to yield an oil that was purified by chromatography on silica gel (EtOAc) to give a white solid (0.161 g, 88%); ¹H NMR (400 MHz, CDCl₃): δ 8.58 (br s, 1H), 7.26 (d, 1H, J=8.7 Hz), 7.05 (d, 1H, J=2.4 Hz), 6.99 (d, 1H, J=2.4 Hz), 6.87 (dd, 1H, J=8.7, 2.4 Hz), 5.83 (br s, 1H), 3.86 (s, 3H), 3.59 (q, 2H, J=6.7 Hz), 2.95 (t, 2H, J=6.7 Hz), 1.93 (s, 3H) ppm; ¹³C NMR (100 MHz, CDCl₃): δ 170.38, 153-97, 131.68, 127.74, 123.00, 112.43, 112.28, 112.15, 100.48, 55.97, 39.87, 25.29, 23.36 ppm; LRMS (ES⁺) calcd for C₁₃H₁₆N₂O₂ [M+H] 233.12 found 233.19.

### 6-methoxy-1-methyl-4,9-dihydro-3H-pyrido[3,4-b]indole (1b)

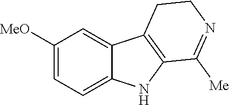 [SMILES: COc1ccc2[nH]c3c(c2c1)CCN=C3C]

To a solution of 1a (0.143 g, 0.57 mmol, 1.0 equiv) in 9:1 EtOAc/CH₃CN (5.8 mL, 0.1 M) was added POCl₃ (0.29 mL, 2.9 mmol, 5.0 equiv) slowly. The solution was heated at reflux for 2 hours, then cooled to 0° C. K₂CO_{3(aq.)} (30% w/w) (20 mL) was then added and the solution was stirred at 0° C. for 1 hour. The organic layer was the collected, and the aqueous layer was extracted with EtOAc (3×20 mL). The combined organic extracts were washed with brine (20 mL), dried over Na₂SO₄, filtered, and concentrated under reduced pressure to yield a residue that was purified by chromatography on silica gel (9:1:0.1 CH₂Cl₂/MeOH/NH₄OH) to give an orange solid (0.073 g, 55%); ¹H NMR (400 MHz, CDCl₃): δ 7.32 (d, H, J=8.7 Hz), 7.01 (d, 1H, 2.3 Hz), 6.98 (dd, 1H, J=8.7, 2.3 Hz), 3.89 (m, 5H), 2.88 (t, 2H, J=8.5 Hz), 2.40 (s, 3H) ppm; ¹³C NMR (100 MHz, CDCl₃): δ 157.88, 154.55, 132.03, 129.71, 125.83, 116.33, 115.74, 112.90, 100.70, 55.79, 48.10, 21.85, 19.43 ppm; LRMS (ES⁺) calcd for C₁₃H₁₄N₂O [M+H] 215.11 found 215.24.

### 6-methoxy-1-methyl-9H-pyrido[3,4-b]indole (1c)

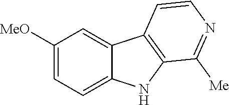 [SMILES: COc1ccc2[nH]c3c(C)nccc3c2c1]

To a solution of 1b (0.057 g, 0.27 mmol, 1.0 equiv) in DMSO (2.0 mL, 0.14 M) was added DBU (0.12 mL, 0.81 mmol, 3.0 equiv). An O₂ balloon was introduced, and the solution was sparged with O₂ for 5 minutes. The solution was then stirred under an O₂ atmosphere at 125° C. for 15 hours. Water was then added (200 mL) and extracted with CH₂Cl₂ (3×20 mL). The combined organic extracts were dried with Na₂SO₄, filtered, and concentrated under reduced pressure to yield a residue that was purified by chromatography on silica gel (95:5 EtOAc/MeOH) to give an orange solid (0.040 g, 71%); ¹H NMR (400 MHz, MeOD): δ 8.12 (d, 1H, J=5.5 Hz), 7.89 (d, 1H, J 5.5 Hz), 7.65 (d, 1H, J=2.4 Hz), 7.49 (d, 1H, J 8.8 Hz), 7.20 (dd, 1H, J=8.8, 2.4 Hz), 3.92 (s, 3H), 2.79 (s, 3H) ppm; ¹³C NMR (100 MHz, MeOD): δ 154.14, 141.72, 136.09, 135.79, 135.42, 128.16, 121.51, 118.35, 112.69, 112.31, 102.77, 54.94, 18.23 ppm; LRMS (ES⁺) calcd for C₁₃H₁₂N₂O [M+H] 213.10 found 213.26.

### 2-(6-methoxy-1-methyl-9H-pyrido[3,4-b]indol-9-yl)-N,N-dimethylethan-1-amine (HTW_A_94)

To 1c (0.185 g, 0.87 mmol, 1.0 equiv) in DMSO (2.2 mL, 0.4 M) was added 2-chloro-N,N-dimethylamino-ethyl hydrochloride (0.138 g, 0.96 mmol, 1.1 equiv), potassium iodide (0.159 g, 0.96 mmol, 1.1 equiv), and potassium hydroxide pellets (0.245 g, 4.36 mmol, 5.0 equiv). The mixture was stirred under a nitrogen atmosphere and monitored by TLC until the starting material was consumed (~48 hours). The solution was then diluted with 1.0 M NaOH_{(aq)} (15 mL) and the aqueous phase was extracted with CH₂Cl₂ (3×10 mL). The combined organic extracts were dried over Na₂SO₄, filtered, and concentrated under reduced pressure to yield an oil that was purified by chromatography on silica gel (9:1:0.1 EtOAc/MeOH/NH₄OH) to give a tan solid (0.127 g, 51%); ¹H NMR (400 MHz, MeOD): δ 8.04 (d, 1H, J=5.3 Hz), 7.76 (d, 1H, J=5.3 Hz), 7.50 (d, 1H, J=2.4 Hz), 7.32 (d, 1H, J=8.9 Hz), 7.25 (dd, 1H, J=8.9, 2.4 Hz), 4.61 (t, 2H, J=8.1 Hz), 3.91 (s, 3H), 2.99 (s, 3H), 2.65 (t, 2H, J=8.1 Hz), 2.37 (s, 6H) ppm; ¹³C NMR (100 MHz, MeOD): δ 154.37, 140.81, 136.48, 136.03, 135.06, 129.07, 121.11, 118.39, 112.95, 110.29, 102.72, 58.05, 54.91, 44.52, 42.36, 21.22 ppm; LRMS (ES⁺) calcd for C₁₇H₂₁N₃O [M+H] 284.17 found 284.36.

### Example 2: 2-(6-methoxy-1-methyl-9H-pyrido[3,4-b]indol-9-yl)-N,N-dimethylethan-1-amine Fumarate Salt (1:2) (HTW_A_94 Fumarate)

Fumaric acid (52 mg, 0.45 mmol, 1.0 equiv) was added to a sealed tube containing acetone (3.5 mL). The solution was carefully heated until the fumaric acid was dissolved. After cooling the solution to room temperature, a solution of HTW_A_94 free base (127 mg, 0.45 mmol, 1.0 equiv) in acetone (1.5 mL) was added, and the mixture was cooled in the freezer overnight. The solid was filtered, washed with acetone, and dried under reduced pressure to yield HTW_A_94 fumarate as the 1:2 salt (118 mg, 51%, 96.5% purity by UHPLC); ¹H NMR (400 MHz, DMSO-d₆): δ 8.21 (d, 1H, J=5.3 Hz), 8.01 (d, 1H, J=5.3 Hz), 7.80 (d, 1H, J=2.5 Hz), 7.68 (d, 1H, J=9.0 Hz), 7.26 (dd, 1H, J=9.0, 2.5 Hz), 6.62 (s, 3H), 4.79 (t, 2H, J=8.0 Hz), 3.87 (s, 3H), 3.00 (s, 3H), 2.92 (t, 2H, J=8.0 Hz); ¹³C NMR (100 MHz, DMSO-d₆): δ 167.20, 154.34, 141.80, 137.40, 136.45, 135.29, 134.83, 128.68, 121.56, 118.63, 113.65, 111.77, 104.04, 56.82, 56.15, 44.40, 41.63, 23.29 ppm; LRMS (ES⁺) calcd for C₁₇H₂₁N₃O [M+H] 284.17 found 284.22.

### Example 3: 2-(7-methoxy-1-methyl-9H-pyrido[3,4-b]indol-9-yl)-N,N-dimethylethan-1-amine (HTW_A_8)

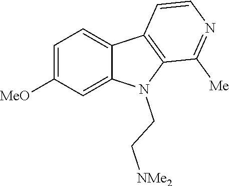 [SMILES: COc1ccc2c3ccnc(C)c3n(CCN(C)C)c2c1]

To harmine (0.050 g, 0.24 mmol, 1.0 equiv) in DMSO (0.6 mL, 0.4 M) was added 2-chloro-N,N-dimethylamino-ethyl hydrochloride (0.037 g, 0.26 mmol, 1.1 equiv), potassium iodide (0.043 g, 0.26 mmol, 1.1 equiv), and potassium hydroxide pellets (0.066 g, 1.2 mmol, 5.0 equiv). The mixture was stirred under a nitrogen atmosphere and monitored by TLC until the starting material was consumed (~48 hours). The solution was then diluted with 1.0 M NaOH_{(aq)} (15 mL) and the aqueous phase was extracted with CH₂Cl₂ (3×10 mL). The combined organic extracts were dried over Na₂SO₄, filtered, and concentrated under reduced pressure to yield an oil that was purified by chromatography on silica gel (gradient 18:1:0.1 EtOAc/MeOH/NH₄OH to 9:1:0.1 EtOAc/MeOH/NH₄OH) to give a tan solid (0.042 g, 63%). ¹H NMR (400 MHz, MeOD): δ 8.10 (d, 1H, J=5.4 Hz), 7.96 (d, 1H, J=8.7 Hz), 7.78 (d, 1H. J=5.4 Hz), 6.97 (d, 1H. J=2.1 Hz), 6.96 (dd, 1H, J=8.7, 2.1 Hz), 4.53 (t, 2H, J=8.0 Hz), 3.93 (s, 3H), 2.95 (s, 3H), 2.63 (t, 2H, J=8.0 Hz), 2.36 (s, 6H) ppm; ¹³C NMR (100 MHz, DMSO-d₆): δ 161.58, 143.33, 140.08, 136.94, 134.91, 129.93, 122.09, 114.53, 112.18, 109.66, 92.57, 57.83, 54.73, 44.52, 42.36, 21.11 ppm; LRMS (ES⁺) calculated for C₁₇H₂₁N₃O [M+H] 284.17 found 284.22.

### Example 4: 2-(7-methoxy-1-methyl-9H-pyrido[3,4-b]indol-9-yl)-N,N-dimethylethan-1-amine Fumarate Salt (2:3) (HTW_A_8 Fumarate)

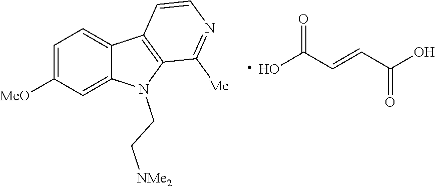 [SMILES: COc1ccc2c3ccnc(C)c3n(CCN(C)C)c2c1 ; O=C(O)/C=C/C(=O)O]

Fumaric acid (41 mg, 0.35 mmol, 1.0 equiv) was added to a sealed tube containing acetone (3.0 mL). The solution was carefully heated until the fumaric acid was dissolved. After cooling the solution to room temperature, a solution of HTW_A_8 free base (100 mg, 0.35 mmol, 1.0 equiv) in acetone (1.0 mL) was added, and the mixture was cooled in the freezer overnight. The solid was filtered, washed with acetone, and dried under reduced pressure to yield HTW_A_8 fumarate as the 2:3 salt (130 mg, 81%, 98.2% purity by UHPLC); ¹H NMR (400 MHz, DMSO-d₆): δ 8.18 (d, 1H, J=5.2 Hz), 8.10 (d, 1H, J=8.6 Hz), 7.90 (d, 1H, J=5.2 Hz), 7.19 (d, 1H, J=2.1 Hz), 6.89 (dd, 1H, J=8.6, 2.1 Hz), 6.62 (s, 3H), 4.71 (t, 2H, J=7.6 Hz), 3.92 (s, 3H), 2.99 (s, 3H), 2.76 (t, 2H, J=7.6 Hz), 2.38 (s, 6H); ¹³C NMR (100 MHz, DMSO-d₆): δ 167.49, 161.29, 143.26, 140.08, 138.00, 134.96, 134.93, 129.37, 123.04, 114.73, 112.88, 110.13, 94.11, 56.55, 56.05, 44.30, 41.43, 23.06 ppm; LRMS (ES⁺) calcd for C₁₇H₂₁N₃O [M+H] 284.17 found 284.22.

### Example 5: N,N-dimethyl-2-(1-methyl-9H-pyrido[3,4-b]indol-9-yl)ethan-1-amine (HTW_A_200)

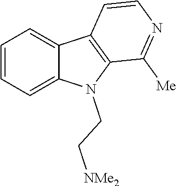 [SMILES: Cc1nccc2c3ccccc3n(CCN(C)C)c12]

### N-(2-(1H-indol-3-yl)ethyl)acetamide (2a)

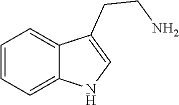 [SMILES: NCCc1c[nH]c2ccccc12]

To a suspension of tryptamine (0.400 g, 2.5 mmol, 1.0 equiv) in DCM (8.5 mL, 0.3 M) was added acetic anhydride (0.29 mL, 3.0 mmol, 1.2 equiv) and triethylamine (0.42 mL, 3.0 mmol, 1.2 equiv). The solution was stirred for 30 minutes at room temperature. Saturated NaHCO_{3(aq.)} (50 mL) was then added, the organic layer was collected, and the aqueous layer was extracted with DCM (2×30 mL). The combined organic extracts were washed with brine (50 mL), dried over Na₂SO₄, filtered, and concentrated under reduced pressure to yield an oil that was purified by chromatography on silica gel (EtOAc) to give a white solid (0.469 g, 93%); ¹H NMR (400 MHz, CDCl₃): δ 9.05 (br s, 1H), 7.63 (d, 1H, J=7.8 Hz), 7.38 (d, 1H, J=7.8 Hz), 7.23 (m, 1H), 7.15 (m, 1H), 6.97 (d, 1H, J=2.0 Hz), 6.09 (br s, 1H), 3.58 (q, 2H. J=6.8 Hz), 3.01 (t, 2H, J=6.8 Hz), 1.95 (s, 3H) ppm; ¹³C NMR (100 MHz, CDCl₃): δ 170.77, 136.61, 127.41, 122.45, 121.97, 119.26, 118.61, 112.48, 111.59, 40.15, 25.29, 23.26 ppm; LRMS (ES⁺) calcd for C₁₂H₁₄N₂O [M+H] 203.11 found 203.23.

### 1-methyl-4,9-dihydro-3H-pyrido[3,4-b]indole (2b)

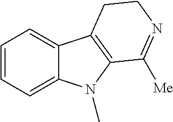

To a solution of 2a (0.942 g, 4.66 mmol, 1.0 equiv) in 9:1 EtOAc/CH₃CN (47 mL, 0.1 M) was added POCl₃ (2.2 mL, 23.3 mmol, 5.0 equiv) slowly. The solution was heated at reflux for 2 hours, then cooled to 0° C. K₂CO_{3(aq.)} (30% w/w) (200 mL) was then added and the solution was stirred at 0° C. for 1 hour. The organic layer was the collected, and the aqueous layer was extracted with EtOAc (3×100 mL). The combined organic extracts were dried over Na₂SO₄, filtered, and concentrated under reduced pressure to yield a residue that was purified by chromatography on silica gel (9:1:0.1 CH₂Cl₂/MeOH/NH₄OH) to give an orange solid (0.757 g, 88%); 1H NMR (400 MHz, CDCl₃): δ 7.64 (d, 1H, J=8.2 Hz), 7.44 (d, 1H, J=8.2 Hz), 7.31 (t, 1H, J=7.6 Hz), 7.18 (t, 1H, J=7.6 Hz), 3.93 (t, 2H, J=8.4 Hz), 2.93 (t, 2H. J=8.4 Hz), 2.40 (s, 3H) ppm; ¹³C NMR (100 MHz, CDCl₃): δ 158.33, 137.03, 129.22, 125.50, 124.55, 120.31, 120.11, 116.66, 112.13, 48.01, 21.97, 19.43 ppm; LRMS (ES⁺) calcd for C₁₂H₁₂N₂ [M+H] 185.10 found 185.21.

### 1-methyl-9H-pyrido[3,4-b]indole (2c)

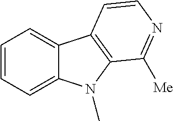

To a solution of 2b (0.720 g, 3.95 mmol, 1.0 equiv) in DMSO (13 mL, 0.3 M) was added DBU (1.8 mL, 11.7 mmol, 3.0 equiv). An O₂ balloon was introduced, and the solution was sparged with O₂ for 5 minutes. The solution was then stirred under an O₂ atmosphere at 125° C. for 72 hours. Water was then added (1000 mL) and extracted with EtOAc (5×100 mL). The combined organic extracts were dried with Na₂SO₄, filtered, and concentrated under reduced pressure. The residue was purified by chromatography on silica gel (95:5 EtOAc/MeOH) to give a brown solid (0.328 g, 46%); ¹H NMR (400 MHz, MeOD): δ 8.17-8.13 (m, 2H), 7.91 (m, 1H), 7.60-7.54 (m, 2H), 7.27-7.24 (m, 1H), 2.81 (s, 3H) ppm; ¹³C NMR (100 MHz, MeOD): δ 141.62, 141.15, 136.37, 134.85, 128.35, 127.98, 121.25, 119.35, 112.68, 111.47, 18.28 ppm; LRMS (ES⁺) calcd for C₁₂H₁₀N₂ [M+H] 183.09 found 183.23.

### N,N-dimethyl-2-(1-methyl-9H-pyrido[3,4-b]indol-9-yl)ethan-1-amine (HTW_A_200)

To 2c (0.208 g, 1.1 mmol, 1.0 equiv) in DMSO (2.9 mL, 0.4 M) was added 2-chloro-N,N-dimethylamino-ethyl hydrochloride (0.181 g, 1.3 mmol, 1.1 equiv), potassium iodide (0.208 g, 1.3 mmol, 1.1 equiv), and potassium hydroxide pellets (0.320 g, 5.7 mmol, 5.0 equiv). The mixture was stirred under a nitrogen atmosphere and monitored by TLC until the starting material was consumed (~48 hours). The solution was then diluted with 1.0 M NaOH_{(aq)} (300 mL) and the aqueous phase was extracted with CH₂Cl₂ (3×100 mL). The combined organic extracts were dried over Na₂SO₄, filtered, and concentrated under reduced pressure to yield a residue that was purified by chromatography on silica gel (9:1:0.1 EtOAc/MeOH/NH₄OH) to give a brown solid (0.059 g, 20%). ¹H NMR (400 MHz, MeOD): δ 8.15-8.12 (m, 2H), 7.91 (d, 1H, J=5.4 Hz), 7.63-7.55 (m, 2H), 7.28 (t, 1H, J=7.9 Hz), 4.62 (t, 2H, J=8.1 Hz), 2.99 (s, 3H), 2.65 (t, 2H, J=8.1 Hz), 2.36 (s, 6H) ppm. ¹³C NMR (100 MHz, MeOD): δ 141.69, 140.91, 136.75, 134.83, 129.57, 128.49, 121.22, 120.96, 119.90, 112.97, 109.51, 57.96, 44.56, 42.35, 21.32 ppm; LRMS (ES⁺) calculated for C₁₆H₁₉N₃ [M+H] 254.16 found 254.25.

### Example 6: N,N-dimethyl-2-(1-methyl-9H-pyrido[3,4-b]indol-9-yl)ethan-1-amine Fumarate Salt (2:3) (HTW_A_200 Fumarate)

Fumaric acid (27 mg, 0.23 mmol, 1.0 equiv) was added to a sealed tube containing acetone (1.0 mL). The solution was carefully heated until the fumaric acid was dissolved. After cooling the solution to room temperature, a solution of HTW_A_200 free base (59 mg, 0.23 mmol, 1.0 equiv) in acetone (1.0 mL) was added, and the mixture was cooled in the freezer overnight. The solid was filtered, washed with acetone, and dried under reduced pressure to yield HTW_A_200 fumarate as the 2:3 salt (55 mg, 66%, >99% purity by UHPLC); ¹H NMR (400 MHz, DMSO-d₆): δ 8.26-8.23 (m, 2H), 8.02 (d, 1H, J=5.2 Hz), 7.75 (d, 1H, J=8.4 Hz), 7.64-7.60 (m, 1H), 7.29 (t, 1H, J=7.4 Hz), 6.62 (s, 3H), 4.81 (t, 2H, J=7.8), 3.03 (s, 3H), 2.92 (t, 2H, J=7.8 Hz), 2.50 (s, 6H) ppm; ¹³C NMR (100 MHz, DMSO-d₆): δ 167.26, 141.77, 141.47, 138.11, 134.90, 134.87, 128.82, 128.80, 122.06, 121.17, 120.33, 113.53, 110.77, 57.15, 44.69, 41.82, 23.46 ppm; LRMS (ES⁺) calcd for C₁₆H₁₉N₃ [M+H] 254.16 found 254.32.

### Example 7: (Z)-1-(3-(2-aminoethyl)-5-methoxybenzo[d]thiazol-2(3H)-ylidene)propan-2-one (3e)

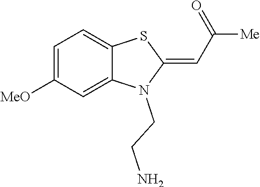 [SMILES: COc1ccc2c(c1)N(CCN)/C(=C/C(C)=O)S2]

### 5-methoxy-3-(2-methoxy-2-oxoethyl)-2-methylbenzo[d]thiazol-3-ium bromide (3a)

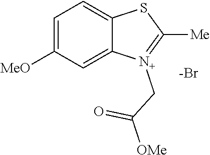 [SMILES: COC(=O)C[n+]1c(C)sc2ccc(OC)cc21]

To a solution of 5-methoxy-2-methylbenzothiazole (0.500 g, 2.79 mmol, 1.0 equiv) in acetonitrile (6 mL, 0.5 M) was added methyl 2-bromoacetate (0.79 mL, 8.4 mmol, 3.0 equiv). The solution was heated to 80° C. and stirred for 48 hours, then cooled to room temperature and diluted with diethyl ether (50 mL). The solid was collected by vacuum filtration and washed with diethyl ether (2×20 mL) to give alight blue solid (0.860 g, 93%); ¹H NMR (400 MHz, MeOD): δ 8.20 (d, 1H, J=9.1 Hz), 7.69 (d, 1H, J=2.3 Hz), 7.45 (dd, 1H. J=9.1, 2.3 Hz), 5.83 (s, 2H), 4.01 (s, 3H), 3.91 (s, 3H), 3.20 (s, 3H) ppm; ¹³C NMR (100 MHz, MeOD): δ 178.63, 165.55, 162.05, 143.19, 124.59, 120.39, 118.57, 99.02, 55.76, 52.83, 49.30, 15.98 ppm; LRMS (ES⁺) calcd for C₁₂H₁₄NO₃S [M+]252.07 found 251.99.

### methyl (Z)-2-(5-methoxy-2-(2-oxopropylidene)benzo[d]thiazol-3(2H)-yl)acetate (3b)

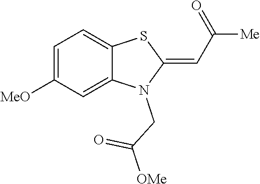 [SMILES: COC(=O)CN1/C(=C/C(C)=O)Sc2ccc(OC)cc21]

To an oven dried flask containing 3a (0.838 g, 2.52 mmol, 1.0 equiv) and MgSO₄ (0.911 g, 7.57 mmol, 3.0 equiv) was added anhydrous CH₃CN (8.4 mL, 0.3 M), acetic anhydride (0.48 mL, 5.05 mmol, 2.0 equiv), and diisopropylethylamine (0.88 mL, 5.05 mmol, 2.0 equiv). The mixture was refluxed under an inert atmosphere for 2 hours before being cooled to room temperature and filtered. The filtrate was diluted with CH₂Cl₂ (40 mL) and poured into saturated NaHCO_{3(aq.)} (200 mL). The organic layer was collected, and the aqueous layer was extracted twice more with CH₂Cl₂ (2×20 mL). The combined organic extracts were washed with brine (100 mL), dried over Na₂SO₄ and concentrated under reduced pressure to yield a solid that was purified by chromatography on silica gel (1.5:1 EtOAc/hexanes) to afford an off-white solid (0.380 g, 51%); ¹H NMR (400 MHz, CDCl₃): δ 7.44 (d, 1H, J=8.5 Hz), 6.77 (dd, 1H, J=8.5, 2.3 Hz), 6.56 (d, 1H, J=2.3 Hz), 5.77 (s, 1H), 4.66 (s, 2H), 3.85 (s, 3H), 3.81 (s, 3H), 2.23 (s, 3H) ppm; ¹³C NMR (100 MHz, CDCl₃): δ 191.50, 167.07, 161.39, 159.50, 140.45, 122.90, 118.36, 109.03, 96.31, 90.84, 55.84, 53.03, 46.78, 29.12 ppm; LRMS (ES⁺) calcd for C₁₄H₁₅NO₄S [M+H] 294.08 found 293.83.

### (Z)-1-(3-(2-hydroxyethyl)-5-methoxybenzo[d]thiazol-2(3H)-ylidene)propan-2-one (3c)

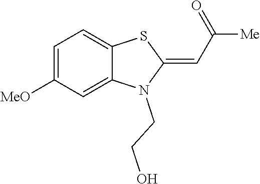 [SMILES: COc1ccc2c(c1)N(CCO)/C(=C/C(C)=O)S2]

To an ice-cold suspension of 3b (0.600 g, 2.05 mmol, 1.0 equiv) in THF (20.5 mL, 0.1 M) was added 4 μM LiBH₄ in THF dropwise (1.03 mL, 4.10 mmol, 2.0 equiv). The solution was warmed to room temperature and stirred for 12 hours. Next, the reaction was quenched with a 10% (v/v) aqueous solution of acetic acid (20 mL) before being diluted with H₂O (150 mL) and extracted with CH₂Cl₂ (3×50 mL). The combined organic extracts were dried over Na₂SO₄ and concentrated under reduced pressure. Addition of EtOAc to the unpurified material resulted in precipitation of 3c, which was collected by vacuum filtration. The mother liquor was concentrated, and this procedure was repeated twice more to afford a white solid (0.369 g, 68%). ¹H NMR (400 MHz, MeOD): δ 7.53 (d, 1H, J=8.5 Hz), 7.05 (d, 1H. J=2.3 Hz), 6.87 (dd, 1H. J=8.5, 2.3 Hz), 6.14 (s, 1H), 4.28 (t, 2H, J=5.6 Hz), 3.94 (t, 2H, J=5.6 Hz), 3.89 (s, 3H), 2.20 (s, 3H) ppm; ¹³C NMR (100 MHz, DMSO-d₆): δ 189.90, 160.61, 159.42, 141.88, 122.96, 117.57, 109.76, 97.85, 90.90, 58.02, 56.13, 48.36, 29.22 ppm; LRMS (ES⁺) calcd C₁₃H₁₅NO₃S for [M+H] 266.09 found 266.13.

### (Z)-1-(3-(2-azidoethyl)-5-methoxybenzo[d]thiazol-2(3H)-ylidene)propan-2-one (3d)

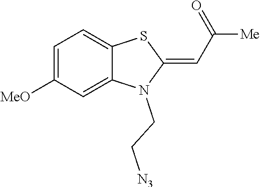 [SMILES: COc1ccc2c(c1)N(CCN=[N+]=[N-])/C(=C/C(C)=O)S2]

To a suspension of 3c (0.163 g, 0.61 mmol, 1.0 equiv) in THF (5 mL) was added PPh₃ (0.177 g, 0.68 mmol, 1.1 equiv) and imidazole (0.046 g, 0.68 mmol, 1.1 equiv). The mixture was cooled to 0° C. before adding 12 (0.312 g, 1.2 mmol, 2.0 equiv). The solution was warmed to room temperature and stirred for 2 hours before adding DMF (5 mL) and NaN₃ (0.200 g, 3.1 mmol, 5.0 equiv). The reaction was monitored by TLC until completion (~24 hours), then diluted with H₂O (500 mL) and extracted with EtOAc (3×100 mL). The combined organic extracts were dried over Na₂SO₄ and concentrated under reduced pressure to yield a solid that was purified by chromatography on silica gel (1.5:1 EtOAc/hexanes) to afford a green solid (0.103 g, 58%). ¹H NMR (400 MHz, CDCl₃): δ 7.44 (d, J=8.5 Hz, 1H), 6.78 (dd, J=8.5, 2.2 Hz, 1H), 6.73 (d, 1H, J=2.2 Hz), 5.88 (s, 1H), 4.14 (t, 2H, J=6.2 Hz), 3.87 (s, 3H), 3.73 (t, 2H, J=6.2 Hz), 2.25 (s, 3H) ppm; ¹³C NMR (100 MHz, CDCl₃): δ 191.27, 161.08, 159.45, 140.54, 122.83, 118.44, 109.13, 96.64, 90.51, 55.85, 47.71, 44.79, 29.11 ppm; LRMS (ES⁺) calcd C₁₃H₁₄N₄O₂S for [M+H] 291.09 found 290.86.

### Z)-1-(3-(2-aminoethyl)-5-methoxybenzo[d]thiazol-2(3H)-ylidene)propan-2-one (3e)

To a solution of 3d (0.125 g, 0.43 mmol, 1.0 equiv) in MeOH (2.9 mL, 0.15 M) was added Pd/C (0.013 g, 10 wt. %). The solution was stirred under an H₂ atmosphere for 12 hours before being filtered through a pad of celite. The filtrate was dried over Na₂SO₄ and concentrated under reduced pressure to yield a solid that was purified by chromatography on silica gel (9:1:0.1 CH₂Cl₂/MeOH/NH₄OH) to afford a yellow solid (0.095 g, 83%). ¹H NMR (400 MHz, MeOD): δ 7.48 (d, 1H, J=8.6 Hz), 6.97 (d, 1H, J=2.2 Hz), 6.82 (dd, 1H, J=8.6, 2.2 Hz), 6.13 (s, 1H), 4.14 (t, 2H, J=7.3 Hz), 3.86 (s, 3H), 2.97 (t, 2H, J=7.3), 2.18 (s, 3H) ppm; ¹³C NMR (100 MHz, MeOD): δ 191.25, 162.65, 159.95, 140.70, 122.34, 117.92, 110.02, 96.40, 89.90, 54.92, 47.30, 37.78, 27.12 ppm; LRMS (ES⁺) calcd for C₁₃H₁₆N₂O₂S [M+H] 265.10 found 265.14.

### Example 8: (Z)-1-(3-(2-(dimethylamino)ethyl)-5-methoxybenzo[d]thiazol-2(3H)-ylidene)propan-2-one Fumarate Salt (1:1) (HTW_B_14 Fumarate)

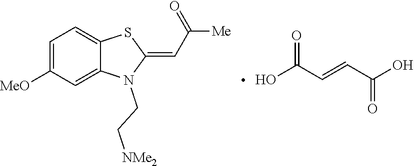 [SMILES: COc1ccc2c(c1)N(CCN(C)C)/C(=C/C(C)=O)S2 ; O=C(O)/C=C/C(=O)O]

To an ice-cold solution of 4e (0.050 g, 0.19 mmol, 1.0 equiv) and glacial acetic acid (0.05 mL, 0.95 mmol, 5.0 equiv) in MeOH (3.8 mL, 0.05 M) was added NaCNBH₃ (0.026 g, 0.42 mmol, 2.2 equiv) followed by 37% formaldehyde_{(aq)} (0.04 mL, 0.49 mmol, 2.6 equiv). The reaction was stirred at room temperature for 3 h before being concentrated under reduced pressure. The residue was then diluted with CH₂Cl₂ (10 mL) and 1 μM NaOH_{(aq)} (25 mL). The phases were separated, and the aqueous phase was extracted with CH₂Cl₂ (3×15 mL). The organic extracts were combined, dried over Na₂SO₄, filtered, and concentrated under reduced pressure. The unpurified material was dissolved in acetone (2.5 mL) and was added to a boiling solution of fumaric acid (0.016 g, 0.14 mmol, 0.8 equiv) in acetone (3 mL). A precipitate formed immediately, and the solution was stored at −20° C. overnight. The solid was filtered, washed with acetone, and dried under reduced pressure to yield HTW_B_14 fumarate as the 1:1 fumarate salt (0.051 g, 66%, >99% purity by UHPLC). ¹H NMR (400 MHz, DMSO-d₆): δ 7.60 (d, 1H, J=8.5 Hz), 7.00 (d, 1H, J=2.3 Hz), 6.80 (dd, 1H, J=8.5, 2.2 Hz), 6.62 (s, 2H), 6.09 (s, 1H), 4.24 (t, 2H, J=7.0 Hz), 3.83 (s, 3H), 2.67 (t, 2H, J=7.0 Hz), 2.10 (s, 3H) ppm; ¹³C NMR (100 MHz, DMSO-d₆): δ 190.07, 167.32, 160.16, 159.58, 141.04, 134.90, 123.25, 117.52, 109.90, 97.35, 90.89, 56.11, 54.33, 45.08, 43.04, 29.24 ppm; LRMS (ES⁺) calcd for C₁₅H₂₀N₂O₂S [M+H] 293.13 found 293.27.

### Example 9: (Z)-1-(3-(2-aminoethyl)-6-methoxybenzo[d]thiazol-2(3H)-ylidene)propan-2-one (4e)

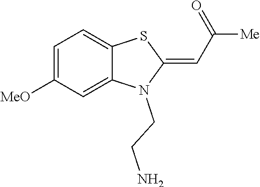 [SMILES: COc1ccc2c(c1)N(CCN)/C(=C/C(C)=O)S2]

### 6-methoxy-3-(2-methoxy-2-oxoethyl)-2-methylbenzo[d]thiazol-3-ium bromide (4a)

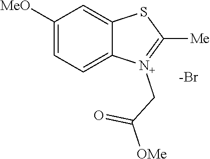 [SMILES: COC(=O)C[n+]1c(C)sc2cc(OC)ccc21]

To a solution of 6-methoxy-2-methylbenzothiazole (0.42 mL, 2.79 mmol, 1.0 equiv) in acetonitrile (6 mL, 0.5 M) was added methyl 2-bromoacetate (0.80 mL, 8.4 mmol, 3.0 equiv). The solution was heated to 70° C. and stirred for 48 hours, then cooled to room temperature and diluted with diethyl ether (50 mL). The solid was collected by vacuum filtration and washed with diethyl ether (2×20 mL) to give a light blue solid (0.668 g, 72%); ¹H NMR (400 MHz, MeOD): δ 8.08 (d, 1H, J=9.4 Hz), 7.89 (s, 1H), 7.48 (d, 1H, 0.1=9.4 Hz), 5.77 (s, 2H), 3.98 (s, 3H), 3.90 (s, 3H), 3.19 (s, 3H) ppm; ¹³C NMR (100 MHz, MeOD): δ 176.09, 165.41, 160.32, 135.59, 130.68, 119.62, 116.91, 105.67, 55.57, 52.82, 49.40, 15.69 ppm; LRMS (ES⁺) calcd for C₁₂H₁₄NO₃S [M+]252.07 found 251.92.

### methyl (Z)-2-(6-methoxy-2-(2-oxopropylidene)benzo[d]thiazol-3(2H)-yl)acetate (4b)

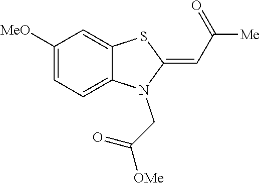 [SMILES: COC(=O)CN1/C(=C/C(C)=O)Sc2cc(OC)ccc21]

To an oven dried flask containing 4a (0.882 g, 2.65 mmol, 1.0 equiv) and MgSO₄ (0.958 g, 7.96 mmol, 3.0 equiv) was added anhydrous CH₃CN (8.8 mL, 0.3 M), acetic anhydride (0.50 mL, 5.31 mmol, 2.0 equiv), and diisopropylethylamine (0.93 mL, 5.31 mmol, 2.0 equiv). The mixture was refluxed under an inert atmosphere for 2 hours before being cooled to room temperature and filtered. The filtrate was diluted with CH₂Cl₂ (40 mL) and poured into saturated NaHCO_{3(aq.)} (200 mL). The organic layer was collected, and the aqueous layer was extracted twice more with CH₂Cl₂ (2×20 mL). The combined organic extracts were washed with brine (100 mL), dried over Na₂SO₄ and concentrated under reduced pressure to yield a solid that was purified by chromatography on silica gel (1.5:1 EtOAc/hexanes) to afford an off-white solid (0.453 g, 58%); ¹H NMR (400 MHz, CDCl₃): δ 7.14 (d, 1H, J=2.0 Hz), 6.93-6.91 (m, 2H), 5.75 (s, 1H), 4.68 (s, 2H), 3.85 (s, 3H), 3.81 (s, 3H), 2.24 (s, 3H) ppm; ¹³C NMR (100 MHz, CDCl₃): δ 191.40, 167.14, 160.46, 156.28, 133.20, 128.25, 113.63, 109.75, 107.14, 90.29, 55.97, 53.01, 46.89, 29.03 ppm; LRMS (ES⁺) calcd for C₁₄H₁₅NO₄S [M+H] 294.08 found 293.83.

### (Z)-1-(3-(2-hydroxyethyl)-6-methoxybenzo[d]thiazol-2(3H)-ylidene)propan-2-one (4c)

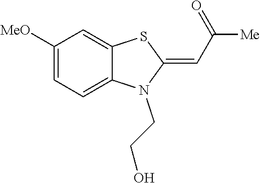 [SMILES: COc1ccc2c(c1)S/C(=C\C(C)=O)N2CCO]

To an ice-cold suspension of 4b (0.410 g, 1.40 mmol, 1.0 equiv) in THF (14.0 mL, 0.1 M) was added 4 μM LiBH₄ in THF dropwise (0.70 mL, 2.80 mmol, 2.0 equiv). The solution was warmed to room temperature and stirred for 12 hours. Next, the reaction was quenched with a 10% (v/v) aqueous solution of acetic acid (20 mL) before being diluted with H₂O (150 mL) and extracted with CH₂Cl₂ (3×50 mL). The combined organic extracts were dried over Na₂SO₄ and concentrated under reduced pressure. Addition of EtOAc to the unpurified material resulted in precipitation of 4c, which was collected by vacuum filtration. The mother liquor was concentrated, and this procedure was repeated twice more to afford a white solid (0.267 g, 72%). ¹H NMR (400 MHz, MeOD): δ 7.35 (d, 1H, J=9.0 Hz), 7.24 (d, 1H. J=2.5 Hz), 6.98 (dd, 1H, J=9.0, 2.6 Hz), 6.06 (s, 1H), 4.24 (t, 2H, J=5.6 Hz), 3.91 (t, 2H. J=5.6 Hz), 3.83 (s, 3H), 2.18 (s, 3H) ppm; ¹³C NMR (100 MHz, MeOD): δ 190.79, 161.89, 156.47, 133.99, 127.89, 113.78, 111.83, 105.87, 89.53, 58.18, 54.95, 48.06, 26.98 ppm; LRMS (ES⁺) calcd for C₁₃H₁₅NO₃S [M+H] 266.09 found 266.06.

### (Z)-1-(3-(2-azidoethyl)-6-methoxybenzo[d]thiazol-2(3H)-ylidene)propan-2-one (4d)

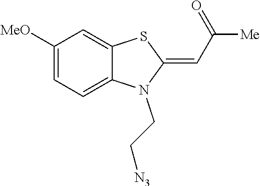 [SMILES: COc1ccc2c(c1)S/C(=C\C(C)=O)N2CCN=[N+]=[N-]]

To a suspension of 4c (0.213 g, 0.80 mmol, 1.0 equiv) in THF (6 mL) was added PPh₃ (0.232 g, 0.88 mmol, 1.1 equiv) and imidazole (0.060 g, 0.88 mmol, 1.1 equiv). The mixture was cooled to 0° C. before adding 12 (0.408 g, 1.61 mmol, 2.0 equiv). The solution was warmed to room temperature and stirred for 2 hours before adding DMF (6 mL) and NaN₃ (0.261 g, 4.01 mmol, 5.0 equiv). The reaction was monitored by TLC until completion (~24 hours), then diluted with H₂O (600 mL) and extracted with EtOAc (3×100 mL). The combined organic extracts were dried over Na₂SO₄ and concentrated under reduced pressure to yield a solid that was purified by chromatography on silica gel (1.5:1 EtOAc/hexanes) to afford a yellow solid (0.136 g, 58%). ¹H NMR (400 MHz, CDCl₃): δ 7.15-7.07 (m, 2H), 6.94 (dd, 1H, J=8.9, 2.5 Hz), 5.84 (s, 1H), 4.16 (t, 2H, J=6.2 Hz), 3.86 (s, 3H), 3.73 (t, 2H, J=6.2 Hz), 2.26 (s, 3H) ppm; ¹³C NMR (100 MHz, CDCl₃): δ 191.13, 160.05, 156.17, 133.26, 128.33, 113.72, 110.31, 106.93, 89.90, 55.95, 47.78, 44.91, 29.02 ppm; LRMS (ES⁺) calcd C₁₃H₁₄N₄O₂S for [M+H] 291.09 found 290.86.

### (Z)-1-(3-(2-aminoethyl)-6-methoxybenzo[d]thiazol-2(3H)-ylidene)propan-2-one (4e)

To a solution of 4d (0.102 g, 0.35 mmol, 1.0 equiv) in MeOH (2.3 mL, 0.15 M) was added Pd/C (0.010 g, 10 wt. %). The solution was stirred under an H₂ atmosphere for 12 hours before being filtered through celite. The filtrate was dried over Na₂SO₄ and concentrated under reduced pressure to yield a solid that was purified by chromatography on silica gel (9:1:0.1 CH₂Cl₂/MeOH/NH₄OH) to afford a yellow solid (0.077 g, 83%). ¹H NMR (400 MHz, MeOD): δ 7.29 (d, 1H, J=8.9 Hz), 7.17 (d, 1H, J=2.5 Hz), 6.94 (dd, 1H, J=9.0, 2.5 Hz), 6.06 (s, 1H), 4.13 (t, 2H, J=7.3 Hz), 3.80 (s, 3H), 2.97 (t, 2H, J=7.3 Hz), 2.17 (s, 3H) ppm; ¹³C NMR (100 MHz, MeOD): δ 190.92, 161.47, 156.45, 133.33, 127.97, 113.82, 111.12, 106.16, 89.34, 54.96, 47.37, 37.92, 27.06 ppm; LRMS (ES⁺) calcd for C₁₃H₁₆N₂O₂S [M+H] 265.10 found 265.28.

### Example 10: (Z)-1-(3-(2-(dimethylamino)ethyl)-6-methoxybenzo[d]thiazol-2(3H)-ylidene)propan-2-one Fumarate Salt (1:1) (HTW_B_26 Fumarate)

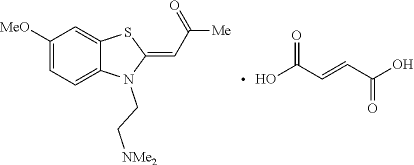 [SMILES: COc1ccc2c(c1)S/C(=C\C(C)=O)N2CCN(C)C ; O=C(O)/C=C/C(=O)O]

To an ice-cold solution of 5e (0.060 g, 0.23 mmol, 1.0 equiv) and glacial acetic acid (0.06 mL, 1.14 mmol, 5.0 equiv) in MeOH (4.5 mL, 0.05 M) was added NaCNBH₃ (0.031 g, 0.50 mmol, 2.2 equiv) followed by 37% formaldehyde_{(aq)} (0.05 mL, 0.59 mmol, 2.6 equiv). The reaction was stirred at room temperature for 3 h before being concentrated under reduced pressure. The residue was then diluted with CH₂Cl₂ (10 mL) and 1 μM NaOH_{(aq)} (30 mL). The phases were separated, and the aqueous phase was extracted with CH₂Cl₂ (3×15 mL). The organic extracts were combined, dried over Na₂SO₄, filtered, and concentrated under reduced pressure. The unpurified material was dissolved in acetone (3.0 mL) and was added to a boiling solution of fumaric acid (0.021 g, 0.18 mmol, 0.8 equiv) in acetone (3 mL). A precipitate formed immediately, and the solution was stored at −20° C. overnight. The solid was filtered, washed with acetone, and dried under reduced pressure to yield HTW_B_26 fumarate as the 1:1 fumarate salt (0.058 g, 63%, >99% purity by UHPLC). ¹H NMR (400 MHz, DMSO-d₆): δ 7.39 (d, 1H. J=2.6 Hz), 7.36 (d, 1H. J=8.9 Hz), 6.98 (dd, 1H, J=8.9, 2.6 Hz), 6.61 (s, 2H), 6.05 (s, 1H), 4.23 (t, 2H, J=7.2 Hz), 3.78 (s, 3H), 2.71 (t, 2H, J=7.2 Hz), 2.38 (s, 6H), 2.09 (s, 3H) ppm; ¹³C NMR (100 MHz, DMSO-d₆): δ 189.80, 167.14, 159.15, 155.96, 134.82, 133.70, 127.62, 114.02, 111.76, 107.51, 90.20, 56.19, 54.49, 45.20, 43.30, 29.14 ppm; LRMS (ES⁺) calcd for C₁₅H₂₀N₂O₂S [M+H] 293.13 found 293.30.

### Example 11: (Z)-1-(3-(2-aminoethyl)benzo[d]thiazol-2(3H)-ylidene)propan-2-one (5e)

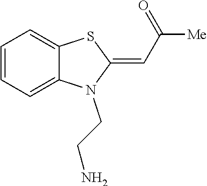 [SMILES: CC(=O)/C=C1\Sc2ccccc2N1CCN]

### 3-(2-methoxy-2-oxoethyl)-2-methylbenzo[d]thiazol-3-ium bromide (5a)

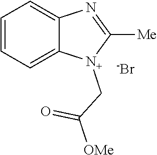

To a solution of 2-methylbenzothiazole (0.2 mL, 1.57 mmol, 1.0 equiv) in acetonitrile (3 mL, 0.5 M) was added methyl 2-bromoacetate (0.44 mL, 4.71 mmol, 3.0 equiv). The solution was heated to 80° C. and stirred for 48 hours, then cooled to room temperature and diluted with diethyl ether (25 mL). The solid was collected by vacuum filtration and washed with diethyl ether (2×20 mL) to give a light blue solid (0.422 g, 89%); ¹H NMR (400 MHz, MeOD): δ 8.37 (d, 1H, J=8.2 Hz), 8.21 (d, 1H, J=8.2 Hz), 7.96-7.92 (m, 1H), 7.89-7.85 (m, 1H), 5.83 (s, 2H), 3.90 (s, 3H), 3.25 (s, 3H) ppm; ¹³C NMR (100 MHz, MeOD): δ 179.30, 165.45, 141.45, 141.45, 129.94, 128.81, 128.59, 124.13, 116.19, 52.90, 49.49, 16.13 ppm; LRMS (ES⁺) calcd for C₁₁H₁₂NO₂S [M+]222.06 found 222.03.

### methyl (Z)-2-(2-(2-oxopropylidene)benzo[d]thiazol-3(2H)-yl)acetate (5b)

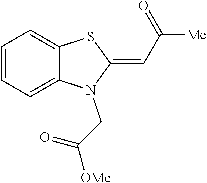 [SMILES: COC(=O)CN1/C(=C/C(C)=O)Sc2ccccc21]

To an oven dried flask containing 5a (0.850 g, 2.81 mmol, 1.0 equiv) and MgSO₄ (1.015 g, 8.44 mmol, 3.0 equiv) was added anhydrous CH₃CN (8.5 mL, 0.3 M), acetic anhydride (0.53 mL, 5.62 mmol, 2.0 equiv), and diisopropylethylamine (0.98 mL, 5.62 mmol, 2.0 equiv). The mixture was refluxed under an inert atmosphere for 2 hours before being cooled to room temperature and filtered. The filtrate was diluted with CH₂Cl₂ (40 mL) and poured into saturated NaHCO_{3(aq.)} (200 mL). The organic layer was collected, and the aqueous layer was extracted twice more with CH₂Cl₂ (2×20 mL). The combined organic extracts were washed with brine (100 mL), dried over Na₂SO₄ and concentrated under reduced pressure to yield a solid that was purified by chromatography on silica gel (1.5:1 EtOAc/hexanes) to afford an off-white solid (0.550 g, 74%); ¹H NMR (400 MHz, CDCl₃): δ 7.60 (d, 1H, J=7.7 Hz), 7.37-7.33 (m, 1H), 7.23-7.18 (m, 1H), 7.03 (d, 1H. J=8.2 Hz), 5.80 (s, 1H), 4.72 (s, 2H), 3.82 (s, 3H), 2.25 (s, 3H) ppm; ¹³C NMR (100 MHz, CDCl₃): δ 191.77, 167.10, 160.30, 139.15, 126.91, 126.52, 123.13, 122.53, 109.18, 90.68, 53.03, 46.73, 29.16 ppm; LRMS (ES⁺) calcd for C₁₃H₁₃NO₃S [M+H] 264.07 found 263.94.

### (Z)-1-(3-(2-hydroxyethyl)benzo[d]thiazol-2(3H)-ylidene)propan-2-one (5c)

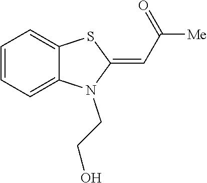 [SMILES: CC(=O)/C=C1\Sc2ccccc2N1CCO]

To an ice-cold suspension of 5b (0.450 g, 1.71 mmol, 1.0 equiv) in THF (17.0 mL, 0.1 M) was added 4 μM LiBH₄ in THF dropwise (0.86 mL, 3.42 mmol, 2.0 equiv). The solution was warmed to room temperature and stirred for 12 hours. Next, the reaction was quenched with a 10% (v/v) aqueous solution of acetic acid (20 mL) before being diluted with H₂O (150 mL) and extracted with EtOAc (3×50 mL). The combined organic extracts were dried over Na₂SO₄ and concentrated under reduced pressure. Addition of CH₂Cl₂ to the unpurified material resulted in precipitation of 5c, which was collected by vacuum filtration. The mother liquor was concentrated, and this procedure was repeated twice more to afford a white solid (0.348 g, 87%). ¹H NMR (400 MHz, MeOD): δ 7.67 (d, 1H, J=7.8 Hz), 7.51-7.38 (m, 2H), 7.24 (ddd, 1H, J=8.1, 7.1, 1.2 Hz), 6.16 (s, 1H), 4.31 (t, 2H, J=5.6 Hz), 3.95 (t, 2H, J=5.6 Hz), 2.21 (s, 3H) ppm; ¹³C NMR (100 MHz, DMSO-d₆): δ 190.11, 159.47, 140.55, 126.80, 126.18, 122.78, 122.51, 111.69, 90.77, 58.02, 48.33, 29.25 ppm; LRMS (ES⁺) calcd for C₁₂H₁₃NO₂S [M+H] 236.07 found 236.16.

### (Z)-1-(3-(2-azidoethyl)benzo[d]thiazol-2(3H)-ylidene)propan-2-one (5d)

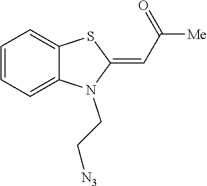 [SMILES: CC(=O)/C=C1\Sc2ccccc2N1CCN=[N+]=[N-]]

To a suspension of 5c (0.138 g 0.59 mmol, 1.0 equiv) in THF (2 mL) was added PPh₃ (0.169 g, 0.65 mmol, 1.1 equiv) and imidazole (0.044 g, 0.65 mmol, 1.1 equiv). The mixture was cooled to 0° C. before adding 12 (0.298 g, 1.17 mmol, 2.0 equiv). The solution was warmed to room temperature and stirred for 2 hours before adding DMF (2.5 mL) and NaN₃ (0.190 g, 2.93 mmol, 5.0 equiv). The reaction was monitored by TLC until completion (~24 hours), then diluted with H₂O (350 mL) and extracted with EtOAc (3×100 mL). The combined organic extracts were dried over Na₂SO₄ and concentrated under reduced pressure to yield a solid that was purified by chromatography on silica gel (1.5:1 EtOAc/hexanes) to afford a yellow solid (0.086 g, 57%). ¹H NMR (400 MHz, CDCl₃): δ 7.58 (dd, 1H, J=8.1, 1.3 Hz), 7.37 (ddd, 1H, J=8.5, 7.3, 1.2 Hz), 7.20 (m, 2H), 5.90 (s, 1H), 4.19 (t, 2H, J=6.2 Hz), 3.74 (t, 2H, J=6.2 Hz), 2.26 (s, 3H) ppm; ¹³C NMR (100 MHz, CDCl₃): δ 191.53, 159.94, 139.22, 126.95, 126.51, 123.03, 122.47, 109.72, 90.34, 47.74, 44.72, 29.16 ppm; LRMS (ES⁺) calcd for C₁₂H₁₂N₄OS [M+H] 261.08 found 260.90.

### (Z)-1-(3-(2-aminoethyl)benzo[d]thiazol-2(3H)-ylidene)propan-2-one (5e)

To a solution of 5d (0.114 g, 0.44 mmol, 1.0 equiv) in MeOH (3.0 mL, 0.15 M) was added Pd/C (0.011 g, 10 wt. %). The solution was stirred under an H₂ atmosphere for 12 hours before being filtered through celite. The filtrate was dried over Na₂SO₄ and concentrated under reduced pressure to afford the product as a yellow solid (0.093 g, 97%). 1H NMR (400 MHz, MeOD): δ 7.62 (d, 1H, J=7.6 Hz), 7.40 (m, 2H), 7.20 (m, 1H), 6.15 (s, 1H), 4.18 (t, 2H, J=7.2 Hz), 2.98 (t, 2H, J=7.2 Hz), 2.19 (s, 3H) ppm; ¹³C NMR (100 MHz, MeOD): δ 191.64, 161.46, 139.44, 126.62, 126.52, 122.86, 121.89, 110.46, 89.80, 47.34, 37.88, 27.19 ppm; LRMS (ES⁺) calcd for C₁₂H₁₄N₂OS [M+H] 235.09 found 235.24.

### Example 12: (Z)-1-(3-(2-(dimethylamino)ethyl)benzo[d]thiazol-2(3H)-ylidene)propan-2-one Fumarate Salt (1:1) (HTW_A_229 Fumarate)

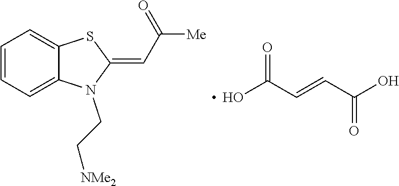 [SMILES: CC(=O)/C=C1\Sc2ccccc2N1CCN(C)C ; O=C(O)/C=C/C(=O)O]

To an ice-cold solution of 6e (0.117 g, 0.50 mmol, 1.0 equiv) and glacial acetic acid (0.15 mL, 2.5 mmol, 5.0 equiv) in MeOH (10 mL, 0.05 M) was added NaCNBH₃ (0.069 g, 1.10 mmol, 2.2 equiv) followed by 37% formaldehyde_{(aq)} (0.10 mL, 1.30 mmol, 2.6 equiv). The reaction was stirred at room temperature for 24 h before being concentrated under reduced pressure. The residue was then diluted with CH₂Cl₂ (25 mL) and 1 μM NaOH_{(aq)} (50 mL). The phases were separated, and the aqueous phase was extracted with CH₂Cl₂ (2×25 mL). The organic extracts were combined, dried over Na₂SO₄, filtered, and concentrated under reduced pressure. The unpurified material was dissolved in acetone (5 mL) and was added to a boiling solution of fumaric acid (0.046 g, 0.40 mmol, 0.8 equiv) in acetone (10 mL). A precipitate formed immediately, and the solution was stored at −20° C. overnight. The solid was filtered, washed with acetone, and dried under reduced pressure to yield HTW_A_229 fumarate as the 1:1 fumarate salt (0.058 g, 31%, >99% purity by UHPLC). ¹H NMR (400 MHz, MeOD): δ 7.73 (d, 1H, J=7.6 Hz, 1H), 7.45-7.37 (m, 2H), 7.19 (td, 1H, J=7.4, 7.0, 1.3 Hz), 6.60 (s, 2H), 6.12 (s, 1H), 4.26 (t, 2H, J=7.2 Hz), 2.70 (t, 2H, J=7.2 Hz), 2.36 (s, 6H), 2.12 (s, 3H) ppm; ¹³C NMR (100 MHz, MeOD): δ 190.36, 167.22, 159.02, 139.75, 134.88, 127.04, 126.22, 123.00, 122.77, 111.11, 90.72, 54.61, 45.34, 43.31, 29.28 ppm; LRMS (ES⁺) calcd for C₁₄H₁₈N₂OS [M+H] 263.12 found 263.23.

### B. Biological Examples

### Example 1: Assay

Data Analysis and Statistics. Treatments were randomized, and data were analyzed by experimenters blinded to treatment conditions. Statistical analyses were performed using GraphPad Prism (version 9.1.3) unless noted otherwise. All comparisons were planned prior to performing each experiment. Data are represented as mean SEM, unless noted otherwise, with asterisks indicating *p<0.05, **p<0.01. ***p<0.001, and ****p<0.0001.

Drugs. Some drugs used in these studies were purchased from commercial sources including harmine hydrochloride (TCI), TG003 (Sigma), and okadaic acid (Cell Signaling). The hemifumarate salt of 5-MeO-DMT was synthesized as described previously.

Animals. Female C57BL/6J mice used in the head twitch response and locomotion assays, and Sprague Dawley dams used to generate embryonic cultures were obtained from Jackson Laboratory (Sacramento, CA). Mice were housed in a temperature- and humidity-controlled room maintained on a 12 h light/dark cycle in groups of four to five (same sex). Animals weighed between 17 and 30 g at the time of the experiments. All experimental procedures involving rodents were approved by the UC Davis Institutional Animal Care and Use Committee (IACUC) and adhered to principles described in the National Institutes of Health Guide for the Care and Use of Laboratory Animals. The University of California, Davis is accredited by the Association for Assessment and Accreditation of Laboratory Animal Care International (AAALAC).

DYRK1A inhibition. IC50s for all compounds at DYRK1A were determined using an ADP-Glo™ assay kit (Promega) and DYRK1A enzyme system (Promega) following the manufacturer's protocol. In brief, 10 μL of DYRKtide substrate (1 mg/mL) and ATP (50 μM) in kinase reaction buffer (40 mM Tris HCl, 20 mM MgCl₂, 0.1 mg/mL BSA, 50 μM DTT in DI water) was added to a solid white 96-well plate. Next, 5 μL of 5× compound solutions in kinase reaction buffer (5% DMSO) or 5% DMSO in kinase reaction buffer was added. Then 10 μL DYRK1A in kinase reaction buffer (2 ng/μL) was added and the plate was incubated at room temperature for 60 min before adding 25 μL ADP-Glo™ reagent. The plate was incubated for 40 min at room temperature. Subsequently, 50 μL of Kinase Detection Reagent was added, the plate was gently shaken for 1 min. and incubated away from light for 30-40 min. Finally, luminescence was measured using a BioTek Synergy HTX multi-mode reader (integration time=1 s). All data was normalized such that the luminescence produced by the lowest concentration treatment of each compound was set to 100% DYRK1A activity.

Kinome Scan. Compound HTW_A_8 fumarate was screened against 97 kinases at a single concentration of 10 μM at Eurofins DiscoverX using their KINOMEscan™ scanEDGE assay. The results are reported as the % activity relative to DMSO control, where lower values indicate higher affinity. See Table 1 for the full kinome profile.

Radioligands Binding Assays. Binding buffer consisted of 50 mM Tris HCl, 10 mM MgCl₂, 0.1 mM EDTA disodium dihydrate in autoclaved deionized water. The solution was adjusted to pH 7.4 with 10 μM NaOH_{(aq)} and stored at 4° C. This solution was used for membrane preparation generation as well as in saturation and competition binding assays.

For the generation of membrane preparations, 15-cm plates of PSYLI2 cells (HEK293T cell line stably expressing psychLight2; mycoplasma free) or HEK293T cells (ATCCCRL-11268; mycoplasma free) lipofected with pCMV-GRAB_5-HT were grown in Dulbecco's Modified Eagle Media (DMEM) (Fisher, 11995073) containing 10% fetal bovine serum (Fisher, 26-140-079) and 1% penicillin/streptomycin (Fisher, 15140-163). When the cells were >90% confluent, the media was removed, and the cells were washed with Dulbecco's Phosphate Buffered Saline (DPBS, 15 mL) (ThermoFisher, #14190-250). Next, DMEM (15 mL) lacking fetal bovine serum and penicillin/streptomycin was added, and the cells were serum-starved for 4-12 h. All but ~5 mL of the media was removed from the plates before placing them on ice. After 1 min on ice, the contents of the plates were scraped and transferred to a pre-chilled 50 mL Falcon tube and then centrifuged (2,000 g, 10 min, 4° C.). The supernatant was discarded, and the pellet was resuspended in ice-cold binding buffer (2 mL per 15-cm plate). The cells in the pellet were lysed by 60 s of repeated pipetting of the suspension with a 10 mL serological pipet, and then the mixture was centrifuged (2,000 g, 10 min, 4° C.). The supernatant was discarded, removing endogenous ligands from the lysed cells, and the pellet was resuspended in ice-cold binding buffer (2 mL per 15-cm plate). The suspension was transferred to 2.0 mL Eppendorf tubes in 1.0 mL aliquots, saving a single aliquot of 0.25-0.50 mL suspension to use for quantification of protein concentration via a Bradford assay. All aliquots were then centrifuged (20,000 g, 20 min, 4° C.). The supernatant was removed, and the aliquots were stored at −80° C. for later use (PSYLI2) or on ice if immediately used (GRAB_5-HT).

For membrane preparation experiments, saturation binding assays to determine the K_{d} values of [³H]-LSD and competition binding assays to determine the Kᵢ values of unlabeled compounds were carried out using Millipore MultiScreen HTS FB filter plates that had been pre-soaked with 0.3% polyethylenimine_{(aq)} (PEI) for at least 30 min on a Millipore Multiscreen HTS vacuum manifold. All compounds and radioligands were diluted in binding buffer. For PSYLI2 cells and HEK293T cells transiently expressing GRAB_5-HT membrane preparation saturation binding experiments, binding buffer (0.21% DMSO, 47.5 μL) was added to half of the wells to determine total binding, while mianserin hydrochloride (0.21% DMSO, 210.5 μM, 47.5 L) was added to the other wells to determine nonspecific binding. [³H]-LSD (20× solution in binding buffer, 5 μL, 0-5.76 μM) was then added to the wells. Membrane preparations were thawed on ice before resuspension in binding buffer. The membrane preparation suspension (47.5 μL, 0.211 μg/μL, 10 g for PSYLI2 or 47.5 μL, 0.526 μg/μL, 25 g for GRAB_5-HT) was added as the final component in each well, bringing the final volume in each well to 100 μL, comprised of either vehicle (0.1% DMSO) or 100 μM mianserin hydrochloride (0.1% DMSO), [³H]-LSD ranging in concentrations from 0-288 nM, and a suspension of membrane preparation protein (10 or 25 g).

For PSYLI2 cells membrane preparation competition binding experiments, [³H]-LSD (2.5× solution in binding buffer, 50 μL, 37.5 nM) and various concentrations of unlabeled compounds (5× solution in binding buffer, 25 μL, 0.5% DMSO, 0-50 M) were added to the wells. Membrane preparations were thawed on ice before being resuspended in binding buffer. The membrane preparation suspension (50 μL, 0.2 μg/μL, 10 μg) was added as the final component in each well, bringing the final volume in each well to 125 μL comprised of [³H]-LSD (15.0 nM), unlabeled compounds (0-10 μM), and a suspension of membrane preparation protein (10 kg).

For HEK293T cells transiently expressing GRAB_5-HT membrane preparation competition binding experiments, [³H]-LSD (2.5× solution in binding buffer, 50 μL, 12.5 nM) and various concentrations of unlabeled compounds (5× solution in binding buffer, 25 μL, 0.5% DMSO, 0-50 μM) were added to the wells. Membrane preparations remained on ice before resuspension in binding buffer. The membrane preparation suspension (50 μL, 0.5 μg/uL, 25 kg) was added as the final component in each well, bringing the final volume in each well to 125 μL comprised of [³H]-LSD (5.00 nM), unlabeled compounds (0-10 μM), and a suspension of membrane preparation protein (25 kg).

All incubations were carried out at ambient temperature for 60 min before separation of bound and free radioligand was conducted via rapid vacuum filtration followed by washing with ice-cold binding buffer (3×200 μL). Dried filter screens from the plates were transferred to 7 mL scintillation vials containing 1 mL PerkinElmer Ultima Gold liquid scintillation cocktail.

Analysis of the radioactivity was conducted using a Beckman LS 6000 liquid scintillation counter. Saturation binding data were analyzed in counts per minute (CPM) using the One site—Total and nonspecific binding nonlinear regression on GraphPad Prism version 9.3.1. Competition binding experiment data were normalized to the radioactivity in disintegrations per minute (DPM) observed in the presence of vehicle (100% [³H]-LSD bound) and 10 μM (R)-DOI (0% [³H]-LSD bound). Competition binding data were analyzed using the One site—Fit Kᵢ nonlinear regression on GraphPad Prism version 9.3.1, constraining HotKdNM to the K_{d} value of the radioligand determined in saturation binding assays, HotNM to the concentration of radioligand employed in the assays, and the bottom plateau to 0%. To analyze the data using this method, the 0.1% DMSO vehicle condition was set to an arbitrarily dilute concentration of 10⁻²⁵ M to be included on the logarithmic scale of the X-axis.

PsychLight. Glass bottom 96-well plates (CellVis) were coated with poly-D-lysine (Sigma, P6407-5MG) overnight at room temperature. Plates were washed with DPBS and PSYLI2 cells were suspended in DMEM containing 10% fetal bovine serum with 5% penicillin-streptomycin and plated at a density of 40,000 cells/well 24 h prior to each experiment. Immediately prior to an experiment, stock solutions of drugs in DMSO (10 mM) were diluted 1:100 in imaging media distributed across an empty 96-well plate (treatment plate) following a randomized plate map. The imaging media consisted of 1×HBSS (Fisher Scientific #14-025-126). Cells grown in a separate 96-well plate (assay plate) were gently washed 3× with imaging media, and the wells were filled with an appropriate volume of imaging media for the respective experiment (vide infra).

For agonist mode experiments, 180 μL of imaging media were added to each well of the assay plate. Wells were then imaged on a CellInsight™ CX7 High Content Analysis imager at 40× with 7 regions of interest (ROI) taken per well with no bias to location and no overlap of the ROIs. Next, 20 μL from the treatment plate was transferred to the assay plate containing a 1:1000 dilution of drug (10 μM as the final concentration in 0.1% DMSO). As positive, negative, and neutral controls, 5-HT (10 μM), ketanserin (10 μM), and DMSO (0.1%) were used, respectively. All final concentrations of drugs were 10 μM (0.1% DMSO) in agonist mode unless stated otherwise. After 5 min of incubation, the same sites were re-imaged using the same settings. Images were processed through a custom Python script to obtain ΔF/F values (https://github.com/moagonzalez/OlsonLab/blob/main/PsychLightV3_Clean.txt). Two replicates from independent seedings were combined and analyzed using GraphPad Prism (v.9.3.1). Data from each replicate were normalized such that 0% ΔF/F was equal to the average of the DMSO treated wells, while 100% ΔF/F was equal to the average of the 5-HT treated wells.

For antagonist mode experiments, 160 mL of imaging media was added to each well of the assay plate. Wells were then imaged on a CellInsight™ CX7 High Content Analysis imager at 40× with 7 ROIs taken per well with no bias to location and no overlap of the ROIs. A 100 μM 5-HT stock solution in DMSO was diluted 1:100 in imaging buffer. Next, 20 μL of this solution was added to the assay plate for a final concentration of 111 nM 5-HT (0.1% DMSO). The same ROIs were imaged after 5 min of incubation. Next, 20 μL from the treatment plate was transferred to the assay plate for a final 1:1000 dilution of drug (10 μM drug, 100 nM 5-HT, 0.2% DMSO). After 5 min of incubation, the same sites were re-imaged using the same settings. Images were processed through a custom Python script to obtain ΔF/F values (https://github.com/moagonzalez/OlsonLab/blob/main/PsychLightV3_Clean.txt). Two replicates from independent seedings were combined and analyzed using GraphPad Prism (v.9.3.1). Data from each replicate were transformed such that 0% ΔF/F was equal to the average of the wells that received 5-HT pretreatment and DMSO posttreatment.

IPOne Assays. The 5-HT2AR and 5-HT2CR IPOne HTRF assays were performed at Epics Therapeutics S.A. (Belgium, FAST-0505I) using conventional methods. Briefly, CHO-K1 cells expressing human recombinant 5-HT2AR or 5-HT2CR grown to mid-log phase in culture media without antibiotics were detached with PBS-EDTA, centrifuged, and resuspended in medium without antibiotics buffer. Then, 20,000 cells were distributed in a 96-well plate and incubated overnight at 37° C. with 5% CO₂. For agonist testing, the medium was removed and 20 μL of assay buffer plus 20 μL of test compound or reference agonist (α-Me-5-HT) was added to each well. The plate was incubated for 60 min at 37° C. with 5% CO₂. For antagonist testing, the medium was removed and 20 μL of assay buffer plus 20 μL of test compound or reference antagonist (ketanserin) was added to each well. The plate was incubated for 60 min at 37° C. with 5% CO₂. After addition of the lysis buffer containing IP1-d2 and anti-IP1 cryptate detection reagents, plates were incubated for 1 h at room temperature and fluorescence ratios were measured according to the manufacturer's specifications using the HTRF kit.

Phospho-Tau Assay Using Cultured Rat Cortical Neurons. Primary cortical cell cultures were prepared as described previously.² Briefly, pregnant Sprague Dawley dams were euthanized at embryonic day 18 (E18), and the cortices of the pups were harvested. Cells were plated on poly-d-lysine coated plates at 15,000 cells per well. Cultures were maintained at 37° C. under an atmosphere containing 5% CO₂. Plating media consisted of 10% heat-inactivated fetal bovine serum (FBS; Fisher) 1% penicillin-streptomycin (Fisher), and 0.5 mM glutamine (Life Technologies) in Neurobasal (Fisher). After 16-24 h, media was exchanged for replacement media consisting of IX B27 supplement (Fisher), 1% penicillin-streptomycin, 0.5 mM glutamine, and 12.5 μM glutamate in Neurobasal. On DIV6 and DIV13, 50% of media was removed, and feeding media containing 1×B27 supplement, 1% penicillin-streptomycin, 0.5 mM glutamine in Neurobasal was added with an additional 20% volume added to account for evaporation. On DIV14, the volume of each well was adjusted to 180 μL. Next, 1000× solutions of compounds in DMSO were diluted 1:100 into Neurobasal and 20 μL of the resulting solutions was added to the appropriate wells (total dilution=1:1000, 0.1% DMSO). For wells that were not treated with compounds, DMSO was diluted 1:100 into Neurobasal and 20 μL of the resulting solution was added to the appropriate wells. All treatments were randomized. After 24 h, 20 μL was removed from each well. A 30 μM okadaic acid solution in DMSO was diluted 1:100 into Neurobasal and 20 μL of the resulting solution was added to the appropriate wells to give a final DMSO concentration of 0.2% For wells that did not receive okadaic acid, DMSO was diluted 1:100 into Neurobasal and 20 μL of the resulting solution was added to the appropriate wells. After 1 h, neurons were fixed by removing 80% of the media and replacing it with a volume of 4% aqueous paraformaldehyde (ThermoFisher) equal to 50% of the working volume of the well. Then, the cells were incubated at room temperature for 15 min before the fixative was aspirated and each well was washed twice with DPBS. Cells were permeabilized using 0.2% Triton X-100 (ThermoFisher) in DPBS for 15 min at room temperature without shaking. Plates were blocked with blocking buffer (2% bovine serum albumin and 0.2 μM glycine in DPBS) overnight at 4° C. Then, plates were incubated overnight at 4° C. with gentle shaking in antibody dilution buffer (ADB) (1% bovine serum albumin and 0.2 μM glycine in DBPS) containing a chicken anti-MAP2 antibody (1:10,000; EnCor, CPCA-MAP2), guinea pig anti-Tau antibody (1:1000; Synaptic Systems), mouse anti-phosphoTau (Ser202/Thr205) antibody (1:1000; Invitrogen), and rabbit anti-phosphoTau (Ser396) antibody (1:1000; Invitrogen). Plates were washed twice with DPBS and once with ADB before being incubated for 1 h at room temperature in ADB containing an anti-chicken IgY secondary antibody conjugated to Alexa Fluor™ Plus 800 (1:1000; Invitrogen), an anti-mouse IgG secondary antibody conjugated to Alexa Fluor™ Plus 680 (1:1000; Invitrogen), an anti-guinea pig IgG secondary antibody conjugated to Alexa Fluor™ 596 (1:1000; Invitrogen), and an anti-rabbit IgG secondary antibody conjugated to Alexa Fluor™ Plus 488 (1:1000; Invitrogen). Plates were washed three times with DBPS, and after the final wash, 100 μL of DPBS was added per well before imaging on a CellInsight™ CX7 High Content Analysis imager at 20× with 12 ROIs taken per well with no bias to location and no overlap of the ROIs. Images were analyzed using Cellnomics Scan software (version 6.6.2). The compartmental analysis assay was used to measure the average intensity of each channel per field and determine the reported intensity ratios.

Spinogenesis. Spinogenesis experiments were performed as previously described with the exception that cells were treated at DIV14 and fixed 24 h after treatment on DIV15. The images were taken on a Nikon HCA confocal microscope with a 100×/NA 1.45 oil objective. Experiments were performed in duplicate with plates prepared using neurons obtained from independent pregnant dams. Images were analyzed by an experimenter blinded to the treatment conditions.

Head-Twitch Response and Locomotion. Mice (9-10 weeks old) were injected intraperitoneally with compounds dissolved in 10% Kolliphor® EL (Sigma) and 10% DMSO in 0.9% saline (injection volume 5 mL/kg) or vehicle, placed in an empty cage, and filmed for 10 min. Locomotion was quantified using ANYmaze Video Tracking System, version 7.07 (Stoelting Co.). After 10 min, mice were then injected with 5-MeO-DMT dissolved in 0.9% saline (10 mg/kg, injection volume 5 mL/kg) or vehicle and filmed for 20 min. Cages were cleaned with 70% ethanol between experiments. The first 10 min of each video was scored for the number of head-twitches by two trained observers blinded to treatment condition (Pearson correlation coefficients=0.91) and these results were averaged. The last 20 min of each video were scored by one trained observer blinded to the treatment conditions.

TABLE 1
Kinome scan data of HTW_A_8 fumarate and harmine

| | Percent Control | |
|---|---|---|
| DiscoveRx Gene Symbol | HTW_A_8 fumarate | Harmine |
| ABL1(E255K)-phosphorylated | 79 | 74 |
| ABL1(T315I)-phosphorylated | 100 | 76 |
| ABL1-nonphosphorylated | 79 | 90 |
| ABL1-phosphorylated | 81 | 100 |
| ACVR1B | 83 | 91 |
| ADCK3 | 100 | 88 |
| AKT1 | 96 | 96 |
| AKT2 | 100 | 80 |
| ALK | 79 | 92 |
| AURKA | 100 | 85 |
| AURKB | 100 | 80 |
| AXL | 97 | 82 |
| BMPR2 | 100 | 96 |
| BRAF | 100 | 100 |
| BRAF(V600E) | 100 | 90 |
| BTK | 100 | 100 |
| CDK11 | 95 | 83 |
| CDK2 | 100 | 98 |
| CDK3 | 91 | 100 |
| CDK7 | 15 | 21 |
| CDK9 | 100 | 67 |
| CHEK1 | 93 | 96 |
| CSF1R | 98 | 88 |
| CSNK1D | 88 | 13 |
| CSNK1G2 | 86 | 19 |
| DCAMKL1 | 98 | 79 |
| DYRK1B | 10 | 6.1 |
| EGFR | 84 | 96 |
| EGFR(L858R) | 87 | 81 |
| EPHA2 | 88 | 93 |
| ERBB2 | 100 | 85 |
| ERBB4 | 100 | 83 |
| ERK1 | 85 | 99 |
| FAK | 100 | 100 |
| FGFR2 | 95 | 91 |
| FGFR3 | 95 | 94 |
| FLT3 | 85 | 85 |
| GSK3B | 100 | 71 |
| IGF1R | 85 | 100 |
| IKK-alpha | 100 | 81 |
| IKK-beta | 100 | 95 |
| INSR | 100 | 100 |
| JAK2(JH1domain-catalytic) | 100 | 92 |
| JAK3(JH1domain-catalytic) | 100 | 61 |
| JNK1 | 100 | 41 |
| JNK2 | 81 | 58 |
| JNK3 | 91 | 80 |
| KIT | 100 | 86 |
| KIT(D816V) | 100 | 91 |
| KIT(V559D, T670I) | 89 | 92 |
| LKB1 | 71 | 91 |
| MAP3K4 | 100 | 93 |
| MAPKAPK2 | 99 | 72 |
| MARK3 | 100 | 91 |
| MEK1 | 87 | 79 |
| MEK2 | 82 | 96 |
| MET | 100 | 100 |
| MKNK1 | 93 | 79 |
| MKNK2 | 100 | 22 |
| MLK1 | 91 | 100 |
| p38-alpha | 100 | 95 |
| p38-beta | 98 | 85 |
| PAK1 | 100 | 73 |
| PAK2 | 96 | 47 |
| PAK4 | 100 | 76 |
| PCTK1 | 96 | 100 |
| PDGFRA | 100 | 90 |
| PDGFRB | 100 | 84 |
| PDPK1 | 76 | 100 |
| PIK3C2B | 100 | 97 |
| PIK3CA | 100 | 91 |
| PIK3CG | 33 | 49 |
| PIM1 | 94 | 45 |
| PIM2 | 84 | 39 |
| PIM3 | 96 | 43 |
| PKAC-alpha | 97 | 88 |
| PLK1 | 100 | 93 |
| PLK3 | 96 | 75 |
| PLK4 | 81 | 77 |
| PRKCE | 100 | 91 |
| RAF1 | 93 | 89 |
| RET | 95 | 91 |
| RIOK2 | 83 | 74 |
| ROCK2 | 73 | 43 |
| RSK2(Kin.Dom.1-N-terminal) | 99 | 72 |
| SNARK | 100 | 95 |
| SRC | 99 | 90 |
| SRPK3 | 81 | 84 |
| TGFBR1 | 90 | 91 |
| TIE2 | 99 | 94 |
| TRKA | 93 | 100 |
| TSSK1B | 100 | 100 |
| TYK2(JH1domain-catalytic) | 97 | 96 |
| ULK2 | 100 | 89 |
| VEGFR2 | 100 | 78 |
| YANK3 | 94 | 77 |
| ZAP70 | 93 | 92 |

^{a}HTW_A_8 fumarate was screened at 10 μM at 97 kinases using the DiscoverX KINOMEscan. Results are reported as percent activity of DMSO control, where lower values indicate higher affinity. Data for harmine is presented as previously reported.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating a brain disorder, comprising administering to a subject in need thereof a therapeutically effective amount of a compound of Formula I or Formula II: 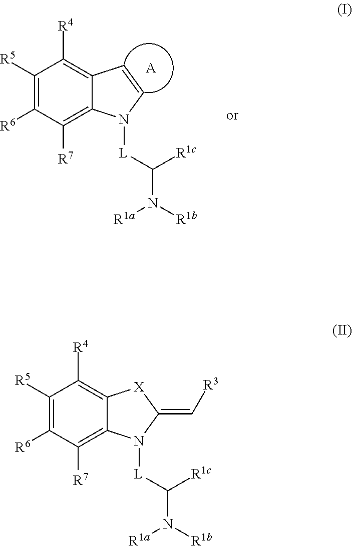
or a pharmaceutically acceptable salt thereof, in an amount sufficient to increase neuronal plasticity of the neuronal cell, wherein:
R^{1a}, R^{1b}, and R^{1c} are each independently hydrogen, C₁₋₆ alkyl, C₃₋₈ cycloalkyl, or C₄₋₁₄ alkyl-cycloalkyl;
alternatively, two of R^{1a}, R^{1b}, and R^{1c} are combined with the atoms to which they are attached to form a C₃₋₁₂ heterocycloalkyl;
Ring A is a 5 to 6 membered heteroaryl ring having 1 to 3 heteroatoms each independently N, O, or S, substituted with 0 to 3 R² groups;
R², R⁴, R⁵, R⁶ and R⁷ are each independently hydrogen, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{8b}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
alternatively, R⁴ and R⁵, R⁵ and R⁶, or R⁶ and R⁷ are combined with the atoms to which they are each attached to form a C₃₋₆ cycloalkyl, C₃₋₆ heterocycloalkyl, C₆₋₁₂ aryl, or C₅₋₁₀ heteroaryl; and
R³ is C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{8a}, —NO₂, —CN, —C(O)R^{Bb}, —C(O)OR^{8b}, —OC(O)R^{8b}, —OC(O)OR^{8b}, —N(R^{8b}R^{8c}), —N(R^{8b})C(O)R^{8c}, —C(O)N(R^{8b}R^{8c}), —N(R^{8b})C(O)OR^{8c}, —OC(O)N(R^{8b}R^{8c}), —N(R^{8b})C(O)N(R^{8c}R^{8d}), —C(O)C(O)N(R^{8b}R^{8c}), —S(O₂)R^{8b}, —S(O)₂N(R^{8b}R^{8c}), C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{8a} is C₃₋₈ cycloalkyl, C₃₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₄₋₁₆ alkyl-heteroaryl;
R^{8b}, R^{8c} and R^{8d} are each independently H or C₁₋₆ alkyl;
X is O or S; and
L is C₁₋₆ alkylene.

2. The method of claim 1, wherein the compound, or a pharmaceutically acceptable salt thereof, wherein the compound has the structure of Formula Ia or Formula IIa:

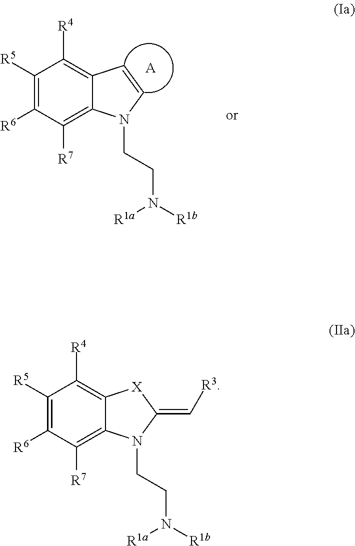

3. The method of claim 1, wherein the compound, or a pharmaceutically acceptable salt thereof, wherein the compound has the structure of Formula Ib or Formula IIb:

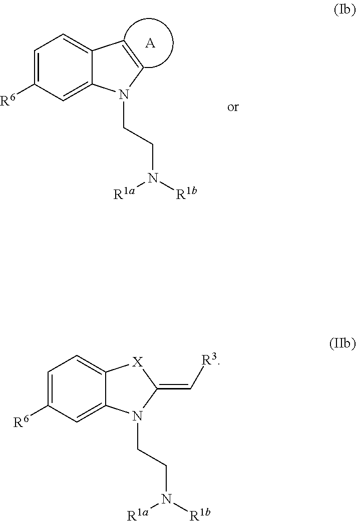

4. The method of claim 1, wherein the compound, or a pharmaceutically acceptable salt thereof, wherein the compound has the structure of Formula Ic or Formula IIc:

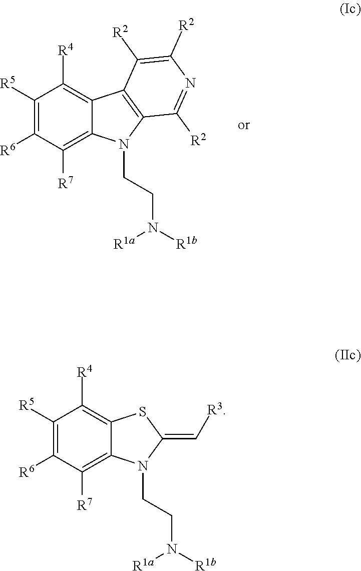

5. The method of claim 1, wherein the compound, or a pharmaceutically acceptable salt thereof, wherein the compound has the structure of Formula Ic-1 or Formula IIc-1:

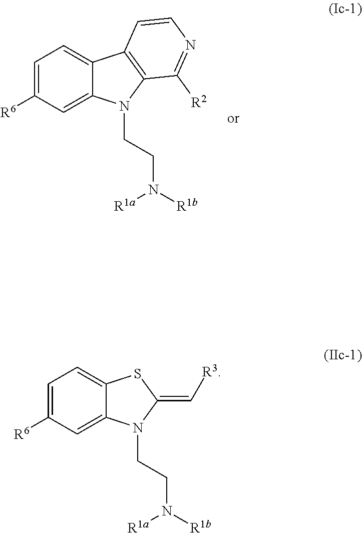

6. The method of any one of claims 1 to 5, wherein the compound, or a pharmaceutically acceptable salt thereof, wherein
R^{1a} and R^{1b} are each C₁₋₆ alkyl;
Ring A is a 6 membered heteroaryl ring having 1 heteroatom of N, substituted with 1 R² groups;
R² is C₁₋₆ alkyl;
R⁴, R⁵, R⁶ and R⁷ are each independently hydrogen, C₁₋₆ alkyl, or C₁₋₆ alkoxy;
R³ is —C(O)R^{8b}, —C(O)OR^{8b}, or —C(O)N(R^{8b}R^{8c});
R^{8b} and R^{8c} are each C₁₋₆ alkyl; and
X is O or S.

7. The method of any one of claims 1 to 6, wherein the compound, or a pharmaceutically acceptable salt thereof, has the structure of:

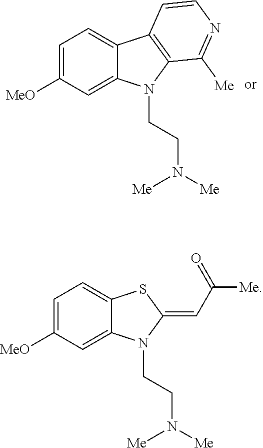 [SMILES: COc1ccc2c3ccnc(C)c3n(CCN(C)C)c2c1 ; "or" ; COc1ccc2c(c1)N(CCN(C)C)/C(=C/C(C)=O)S2]

8. The method of any one of claims 1 to 7, wherein the compound, or a pharmaceutically acceptable salt thereof, has the structure of:

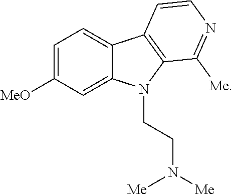 [SMILES: COc1ccc2c3ccnc(C)c3n(CCN(C)C)c2c1]

9. The method of any one of claims 1 to 8, wherein the compound has the structure of:

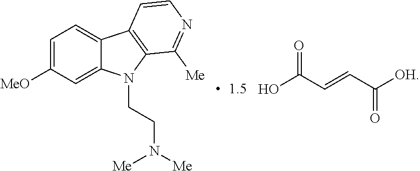 [SMILES: COc1ccc2c3ccnc(C)c3n(CCN(C)C)c2c1 ; O=C(O)/C=C/C(=O)O]

10. The method of any one of claims 1 to 7, wherein the compound, or a pharmaceutically acceptable salt thereof, has the structure of:

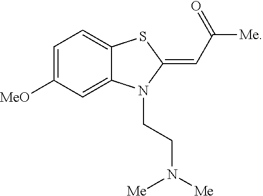 [SMILES: COc1ccc2c(c1)N(CCN(C)C)/C(=C/C(C)=O)S2]

11. The method of any one of claims 1 to 7, wherein the compound, has the structure of:

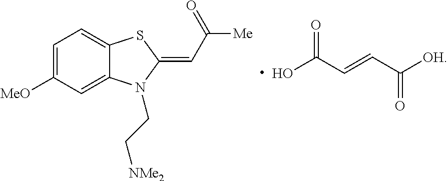 [SMILES: COc1ccc2c(c1)N(CCN(C)C)/C(=C/C(C)=O)S2 ; O=C(O)/C=C/C(=O)O]

12. The method of any one of claims 1 to 11, wherein the compound of Formula (I) and Formula (II) are each present in a sub-therapeutic amount.

13. The method of any one of claims 1 to 12, wherein the brain disorder is a neurodegenerative disorder, Alzheimer's, or Parkinson's disease.

14. The method of any one of claims 1 to 12, wherein the brain disorder is Down's syndrome.

15. The method of any one of claims 1 to 12, wherein the brain disorder is a psychological disorder, depression, addiction, anxiety, or a post-traumatic stress disorder.

16. The method of claim 15, wherein the brain disorder is depression.

17. The method of claim 15, wherein the brain disorder is addiction.

18. The method of claim 15, wherein the brain disorder is treatment resistant depression, suicidal ideation, major depressive disorder, bipolar disorder, schizophrenia, or substance use disorder.

19. The method of any one of claims 1 to 12, wherein the brain disorder is stroke or traumatic brain injury.

20. The method of any one of claims 1 to 19, comprising administering one or more additional therapeutic agent that is lithium, Olanzapine (Zyprexa), Quetiapine (Seroquel), Risperidone (Risperdal), Ariprazole (Abilify), Ziprasidone (Geodon), Clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

21. A method of treating a brain disorder, comprising administering to a subject in need thereof a therapeutically effective amount of a DYRK1A modulator and a 5-HT2 modulator, wherein each modulator is present in a sub-therapeutic amount.

22. A method of treating a brain disorder, comprising administering to a subject in need thereof a therapeutically effective amount of a compound having a DYRK1A binding moiety and a 5-HT binding moiety.

23. The method of claim 21 or 22, wherein the brain disorder is a neurodegenerative disorder, Alzheimer's, or Parkinson's disease.

24. The method of claim 21 or 22, wherein the brain disorder is Down's syndrome.

25. The method of claim 21 or 22, wherein the brain disorder is a psychological disorder, depression, addiction, anxiety, or a post-traumatic stress disorder.

26. The method of claim 25, wherein the brain disorder is depression.

27. The method of claim 25, wherein the brain disorder is addiction.

28. The method of claim 25, wherein the brain disorder is treatment resistant depression, suicidal ideation, major depressive disorder, bipolar disorder, schizophrenia, or substance use disorder.

29. The method of claim 21 or 22, wherein the brain disorder is stroke or traumatic brain injury.
